(19) United States
(12) Patent Application Publication
(10) Pub. No.: US 2015/0366695 A1
(43) Pub. Date: Dec. 24, 2015

(54) LIMB STABILIZATION DEVICE

(71) Applicants: President and Fellows of Harvard College, Cambridge, MA (US); Soft Robotics, Inc., Brookline, MA (US)

(72) Inventors: Kevin C. GALLOWAY, Somerville, MA (US); Jobim SANTOS, Cambridge, MA (US); Ryan KNOPF, Cambridge, MA (US); Joshua Aaron LESSING, Cambridge, MA (US); Mitchell R. ZAKIN, Andover, MA (US); Carl E. VAUSE, Concord, MA (US)

(21) Appl. No.: 14/745,932

(22) Filed: Jun. 22, 2015

Related U.S. Application Data
(60) Provisional application No. 62/014,899, filed on Jun. 20, 2014.

(51) Int. Cl. A61F 5/01

(52) U.S. Cl. CPC A61F 5/012; A61F 5/0106; A61F 5/0111

(57) ABSTRACT

A limb stabilization device is described, including: two or more collars configured to surround a limb and apply a pressure to the limb at or below a threshold pressure, wherein the collar comprises a pressurized bladder or a compressed memory foam to conform the collar to the limb; at least one beam connecting the two or more collars to support the limb; and optionally a pressure modulator configured to regulate the pressure of the bladder to be at or below a threshold pressure.

### RELATED APPLICATIONS

This application claims the priority and benefits to U.S. Provisional Application No. 62/014,899, filed Jun. 20, 2014, the entire content of which is expressly incorporated by reference.

### GOVERNMENT FUNDING CLAUSE

This invention was made with support from the United States government under Grant No. N66001-13-C-4036 awarded by DARPA. The United States government has certain rights to this invention.

### FIELD OF THE INVENTION

The present disclosure generally relates to the field of splinting and limb stabilization device.

### BACKGROUND

An important step in treating any fracture is to stabilize the limb. This step is essential to prevent the fracture site from causing vascular damage until the patient can receive definitive care. The traditional methods for stabilizing a fractured limb are splints, which are typically used in austere environments, and plaster casts, which are typically applied in the hospital. These methods are effective at stabilizing the limb; however, improper application and/or poor monitoring can cause serious problems. For example, splints can cause pressure points, which over time (on the order of hours) can turn into ulcers and necrotic tissue. Alternatively, swelling of the fracture site combined with tight bandages under splints or casts can increase compartmental pressure and increase the risk for compartment syndrome. A patient can suffer permanent muscle and nerve damage and likely require amputation if the compartmental pressure is not released in a timely manner (<6 hrs). While proper application of a limb stabilization device is important for patient outcome, speed of application is important to the medical response team. In battlefield situations and other time sensitive scenarios (e.g., natural disasters), the time spent stabilizing one patient is time that could be spent providing medical attention to another. Furthermore, the more time a medical response team is left exposed on the battlefield or in a hazardous environment, the greater the chance that they too maybe be wounded, or worse, killed.

Thus, there remains a need for limb stabilization devices that are safe, portable, and easy and quick to deploy.

### SUMMARY

Described herein are limb stabilization devices that satisfy the following functional requirements: portable, rapidly deployable, and safe (i.e., can accommodate changes in limb size due to swelling and can anchor to the limb without creating pressure points or constricting blood flow). In some embodiments, three different limb stabilization devices are described.

As used herein, the term “beam,” “stiff beam,” and “stiff beam element” are used interchangeably. A beam is a structural element capable of supporting load primarily by resisting bending. A beam's resistance to bending or stiffness is a function of the shape of the beam's cross-section, length, and materials. As used herein, “splint,” “splint device,” and “limb stabilization device” are used interchangeably.

Unless otherwise defined, used or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially, though not perfectly pure, as practical and imperfect realities may apply; e.g., the potential presence of at least trace impurities (e.g., at less than 1 or 2%) can be understood as being within the scope of the description; likewise, if a particular shape is referenced, the shape is intended to include imperfect variations from ideal shapes, e.g., due to manufacturing tolerances. Percentages or concentrations expressed herein can represent either by weight or by volume.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments. Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise. Additionally, the terms, “includes,” “including,” “comprises” and “comprising,” specify the presence of the stated elements or steps but do not preclude the presence or addition of one or more other elements or steps.

In one aspect, a limb stabilization device is described, including:
two or more collars configured to apply a pressure to a limb at or below a threshold pressure, wherein the collar comprises a pressurizable bladder and/or a compressed memory foam to conform the collar to the limb; and
at least one beam connecting the two or more collars to support the limb.

In any of the preceding embodiments, the limb stabilization device further includes a pressure modulator for regulating the pressure of the bladder to be at or below the threshold pressure.

In any of the preceding embodiments, the pressure modulator is a check valve.

In any of the preceding embodiments, the bladder is in fluidic connection with a gas or fluid pressurization source.

In any of the preceding embodiments, the gas or fluid pressurization source is a gas or fluid hand pump, a compressed fluid or gas cartridge, or a fluid or gas source generated by a chemical reaction.

In any of the preceding embodiments, the threshold pressure is less than or equal to 1 psi.

In any of the preceding embodiments, the collar has a built-in slack to result in an adjustable circumference of the collar based on the size of the limb.

In any of the preceding embodiments, the built-in slack is released after the collar has been applied to the limb.

In any of the preceding embodiments, the built-in slack is created by a connection means connecting two non-adjacent portions of the collar.

In any of the preceding embodiments, the limb stabilization device further including a pinch valve openable when the built-in slack is released.

In any of the preceding embodiments, the collar comprises a channel to receive the beam.

In any of the preceding embodiments, the collar is connected to the beam via hook and loop or the collar is mounted onto the beam via one more optionally detachable mount.

In any of the preceding embodiments, the collar is positioned to compress a wound on the limb.

In any of the preceding embodiments, the collar is positionable along the length of the beam.

In any of the preceding embodiments, the beam is an inflatable rigidizing beam.

In any of the preceding embodiments, the collars and beam are connected to the same or different fluid pressurization source.

In any of the preceding embodiments, the fluid pressurization source is a hand pump or a compressed fluid or gas cartridge.

In any of the preceding embodiments, the limb stabilization device further includes a check valve for controlling the degree of inflation and internal pressure of the beam.

In any of the preceding embodiments, the limb stabilization device further includes a locking mechanism to maintain the beam in a stiff state.

In any of the preceding embodiments, the stiffness of the beam is adjustable by a squeezing force applied to the beam.

In any of the preceding embodiments, the beam and/or collars are made from one or more material capable of being cut, rolled, and/or folded.

In any of the preceding embodiments, the memory foam comprises more than one layer of foam, more than one type of foam, or a combination thereof.

In any of the preceding embodiments, the memory foam is vacuum sealed in the bladder.

In any of the preceding embodiments, the memory foam is maintained in a compressed state by the vacuum seal and the vacuum seal is releasable after the collar is applied to the limb.

In any of the preceding embodiments, the memory foam is exposed to the environment and held in a compressed state by a stretchable structural element or a stain limiting element.

In any of the preceding embodiments, the collar has a high coefficient of friction with skin, is breathable, comprises one or more blood-clotting materials, and/or is fluid-absorbent.

In any of the preceding embodiments, the limb stabilization device further includes a medicine integrated in the collar and/or the beam.

In any of the preceding embodiments, the collar and/or beam comprises one or more capsules containing the medicine and puncturable for delivery into the patient.

In any of the preceding embodiments, the collar comprises more than one independently controlled bladder.

In any of the preceding embodiments, the collar comprises a foam liner.

In another aspect, a limb stabilization device is described, including: one bending actuator or two or more interdigitating bending actuators configured to apply a pressure to the limb at or below than a threshold pressure, wherein the actuator comprises a pressurized bladder and/or a compressed memory foam to cause the actuator to bend to conform to the limb.

In yet another aspect, a limb stabilization device is described, including:
two or more interdigitating bending actuators each comprising a pressurizable bladder and/or a compressed memory foam and configured to bend along a first direction towards the limb in an interdigitated manner upon actuation to apply a pressure to the limb at or below a threshold pressure, wherein the interdigitating bending actuator is actuatable by bladder pressurization or decompression of the memory foam.

In any of the preceding embodiments, the limb stabilization device further includes a pressure modulator for regulating the pressure of the bladder to be at or below a threshold pressure.

In any of the preceding embodiments, the limb stabilization device further comprises a beam and the interdigitating bending actuators are connected to the beam.

In any of the preceding embodiments, the limb stabilization device is made from one or more materials capable of being rolled or folded in the unpressurized state.

In any of the preceding embodiments, the two or more interdigitating bending actuators wrap around the limb upon actuation.

In any of the preceding embodiments, the threshold pressure is equal to or below 1 psi.

In any of the preceding embodiments, after actuation, at least one of the interdigitating bending actuator is bendable along a second direction away from the limb.

In any of the preceding embodiments, the bladder is in fluidic communication with a hand pump or a pressurized fluid or gas cartridge.

In any of the preceding embodiments, the interdigitating bending actuator comprises a memory foam liner.

In any of the preceding embodiments, the interdigitating bending actuator has a high coefficient of friction with skin, is breathable, comprises one or more blood-clotting materials, and/or is fluid-absorbent.

In yet another aspect, a limb stabilization device is described, including:
a bending actuator comprising a plurality of sequentially-disposed pressurizable bladders each in fluidic communication with a fluid pressurization source or enclosing a compressed memory foam; wherein upon actuation, the adjacent bladders expand against each other so that the bending actuator bends along a first direction towards the limb to apply a pressure to the limb at or below a threshold pressure; wherein the bending actuator is actuatable by bladder pressurization or decompression of the memory foam.

In any of the preceding embodiments, the limb stabilization device further includes a pressure modulator for regulating the pressure of the bladder to be at or below a threshold pressure.

In any of the preceding embodiments, the limb stabilization device further comprises a beam and the bending actuator is connected to the beam.

In any of the preceding embodiments, the bending actuator is a bellow bending actuator.

In any of the preceding embodiments, the limb stabilization device is made from one or more materials capable of being rolled or folded in the unpressurized state.

In any of the preceding embodiments, the limb stabilization device wraps around the limb upon actuation.

In any of the preceding embodiments, the threshold pressure is equal to or below 1 psi.

In any of the preceding embodiments, after actuation, the bending actuator is bendable along a second direction away from the limb.

In any of the preceding embodiments, the bending actuator comprises a memory foam liner.

In any of the preceding embodiments, the bending actuator has a high coefficient of friction with skin, is breathable, comprises one or more blood-clotting materials, and/or is fluid-absorbent.

In any of the preceding embodiments, the limb comprises a joint.

In any of the preceding embodiments, the upon actuation, the bending actuator generate forces to move the joint in one or multiple directions.

In any of the preceding embodiments, the limb stabilization device further includes an inertial measurement unit for recording the angle and motion of the joint and/or a computer medium for storing the angle and motion of the joint in a digital database.

In yet another aspect, a limb stabilization device is described, including:
a conformal material layer configured to wrap around a limb and apply a pressure to the limb at or below a threshold pressure, wherein the conformal material layer comprises a pressurizable bladder and/or a compressed memory foam to conform the conformal material layer to the limb; and
optionally at least one beam connected to the conformal material layer to support the limb.

In any of the preceding embodiments, the limb stabilization device further includes a pressure modulator for regulating the pressure of the bladder to be at or below the threshold pressure.

In any of the preceding embodiments, the pressure modulator is a check valve.

In any of the preceding embodiments, the threshold pressure is equal to or below 1 psi.

In any of the preceding embodiments, the beam is connected to the conformal material layer via hook and loop or the beam is connected to the conformal material layer via one or more optionally detachable mount.

In yet another aspect, a method of stabilizing an injured limb is described, including:
providing a limb stabilizing device of any one of the embodiments described herein, supporting the limb using the limb stabilizing device; and
pressurizing the bladder and/or releasing the compressed memory foam to conform the collar to the limb.

In any of the preceding embodiments, the method further includes stabilizing and/or healing the limb.

It is contemplated that any embodiment disclosed herein may be properly combined with any other embodiment disclosed herein. The combination of any two or more embodiments disclosed herein is expressly contemplated.

### BRIEF DESCRIPTION OF DRAWINGS

FIG. 1 depicts a perspective view of a leg splint with inflatable collars that anchor to the leg and stiff beams that bridge the collars to support the leg.

FIG. 2A depicts a cross-section exploded side view of an inflatable collar.

FIG. 2B depicts a cross-section assemble side view of an inflatable collar.

FIG. 2C depicts an isometric view of the inflatable collar—the surface that faces the wearer.

FIG. 2D depicts an isometric view of the inflatable collar—the outside surface.

FIG. 2E depicts an isometric view of the inflatable collar where a small section is folded over to create built-in slack.

FIG. 2F depicts an isometric view of the inflatable collar wrapped around an object.

FIG. 2G depicts an isometric view of the inflatable collars wrapped around an object the built-in slack released.

FIG. 2H depicts an isometric view of the inflatable collar wrapped around an object with the bladder inflated and conforming to the object.

FIG. 3A presents an isometric view of the pressurized fluid connection with a pinch valve mechanism preventing the collar from inflating.

FIG. 3B presents an isometric view of the pinch valve released.

FIG. 4 presents a sample plot of the compressive stress vs. compressive strain of memory foam.

FIG. 5A presents another embodiment of the collar where a foam expanding inside the bladder is used as a method to conform and anchor to a limb.

FIG. 5B depicts the foam in a compressed state inside the bladder and held in this state by negative pressure.

FIG. 6A presents a collar where a foam is not contained in a bladder.

FIG. 6B presents the foam-based collar attached to a representative limb and the distribution of forces.

FIG. 7 depicts multiple inflatable collars capable of being positioned along the length of a stiff or rigidizing beam.

FIG. 8A presents an isometric exploded view of a splint where detachable mounts are used to connect a stiff beam to the collars.

FIG. 8B presents the assembled isometric view where detachable mounts are used to connect a stiff beam to the collars.

FIG. 9A depicts a physical embodiment of the leg splint contained in a small package.

FIG. 9B depicts the splint removed from the package.

FIG. 9C depicts the collars unrolled.

FIG. 9D depicts a physical embodiment of FIG. 7 where the collar positions can be adjusted.

FIG. 9E depicts the collars being fastened to the model leg.

FIG. 9F depicts a top view of the unpressurized device attached to the model leg.

FIG. 9G presents a close-up view of a collar where the built-in slack has not been released.

FIG. 9H presents a close-up view of a collar with the built-in slack released and the pinch valve mechanism disengaged.

FIG. 9I depicts the built-in slack being released on neighboring collars.

FIG. 9J present a view of the device with the built-in slack released from all the collars as indicated by the gap between the skin and the collar's inner surface.

FIG. 9K presents a view of the device where an additional rigidizing strip is attached to increase leg support.

FIG. 9L presents an unpressurized view of the device.

FIG. 9M presents a view of the device when it is pressurized.

FIG. 10 presents a proposed plumbing arrangement for pressuring the splint components.

FIG. 11 depicts a collar that applies compression to a wound.

FIG. 12A presents the splint attached to a patient with the wound exposed.

FIG. 12B presents a wound compression device that can be added separately and anchored to the stiff beams.

FIG. 13A presents a medicinal feature in the form of a medicinal capsule that can be incorporated into the device.

FIG. 13B presents a method for delivering the contents of the medicinal capsule into the patient.

FIG. 13C presents an array of medicinal capsules with a variety of treatments all within a single structure.

FIG. 14A depicts a multi-chamber inflatable collar.

FIG. 14B depicts a multi-chamber inflatable collar used to realign bone.

FIG. 14C depicts a fractured limb.

FIG. 14D depicts a multi-chamber inflatable collars connected by stiff beams to realign the fractured limb.

FIG. 15A presents the unpressurized state of an interdigitating inflatable splint.

FIG. 15B presents the pressurized stage of the interdigitating splint conforming to the patient's limb.

FIG. 15C demonstrates the back-drivability of the device where one of the digits can be pulled back for adjustment or wound inspection.

FIG. 16A presents an exploded view of a bladder used to make a bending actuator.

FIG. 16B presents an assembled view of the bladder.

FIG. 16C depicts a side of view of the bladder attached to a strain limiting layer.

FIG. 16D presents an isometric view of the bladder attached to the strain limiting layer.

FIG. 16E presents a top view of the bladder attached to the strain limiting layer.

FIG. 16F presents an isometric view of the bladder inflated creating a bending motion.

FIG. 17A presents an isometric view of bellowed bending actuator.

FIG. 17B presents a top view of a bellowed bending actuator.

FIG. 17C presents a cross-sectional side view of a bellowed bending actuator.

FIG. 17D presents two bellowed bending actuators integrated together.

FIG. 17E presents two bellowed bending actuators integrate together in a pressurized state.

FIG. 17F presents an exploded view of a bladder of a bending actuator.

FIG. 18A presents the soft bending actuator applied to the ankle with an elastic band to support extension.

FIG. 18B depicts the soft bending actuator pressurized to support ankle flexion.

FIG. 18C depicts a physical embodiment of the bending actuator applied to the ankle in the unpressurized state.

FIG. 18D depicts a physical embodiment of the bending actuator pressurized to support ankle flexion.

FIG. 19A depicts an antagonistic arrangement of soft bending actuators applied to the knee where the actuator located behind the knee can be activated to straighten the leg.

FIG. 19B depicts the actuator bend behind the knee deactivated while the actuator over the knee is activated to support knee bending.

FIG. 20A depicts an exploded isometric view of an inflatable bandage.

FIG. 20B depicts an assembled isometric view of an inflatable bandage.

FIG. 20C depicts the inflatable bandage in a rolled state.

FIG. 20D depicts the inflatable bandage applied to an injured leg.

FIG. 20E depicts stiff strips applied to the inflatable bandage to increase leg support.

FIG. 21A presents a cross-section side view of a rigidizing beam in its flexible state.

FIG. 21B presents a cross-section side view of the rigidizing beam in its rigidized state.

FIG. 21C presents physical embodiment of a beam segment in its flexible state.

FIG. 21D presents a physical embodiment of a beam segment in its rigidized state.

FIG. 21E presents an isometric view with a cut away to show the internal components of the beam in the rigidized state.

FIG. 22 illustrates equations and graph used to calculate the length of built-in slack according to one or more embodiments.

### DETAILED DESCRIPTION

As described herein, a limb stabilization device is disclosed, including two or more collars configured to surround a limb and apply a pressure to the limb at or below a threshold pressure, wherein the collar comprises a pressurized bladder and/or a compressed memory foam to conform the collar to the limb; at least one beam connecting the two or more collars to support the limb. In some embodiments, a pressure modulator configured to regulate the pressure of the bladder to be at or below a threshold pressure can be used. In some embodiments, the pre-compressed state of the memory foam is controlled so that, when released, the memory foam applies a pressure not exceeding a threshold pressure.

In certain embodiments, a pressure modulator or a pressure regulator is a device which can automatically cuts off the flow of a fluid (liquid or gas) at a certain pressure. In certain embodiments, a pressure modulator includes a restricting element, a loading element, and a measuring element. The restricting element is a valve that can provide a variable restriction to the flow, such as a globe valve, butterfly valve, poppet valve, etc. The loading element is a part that can apply the needed force to the restricting element. This loading can be provided by a weight, a spring, a piston actuator, or the diaphragm actuator in combination with a spring. The measuring element functions to determine when the inlet flow is equal to the outlet flow. In certain specific embodiments, the pressure modulator is a check valve. Any other pressure modulator known in the art can be used.

In some embodiments, the limb stabilization device comprises a compressed memory foam or a bladder pressurizable by a fluid or gas to apply a pressure to the limb. The pressures applied do not exceed a threshold pressure. In some embodiments, the threshold pressure is the pressure which does not result in detrimental effects to the patient's limb. Such detrimental effects may include, but are not limited to, restriction to the blood flow, compartmentalized blood flow, and permanent muscle and/or nerve damages. In certain embodiments, the threshold pressure is 1, 0.9, 0.8, 0.7, 0.6, 0.5 psi, or in a range bounded by any two values of pressure described herein. For example, the blood pressure in a single capillary can range from 12 mm Hg (0.23 psi) to 32 mm Hg (0.62 psi) between the venous and arterial ends, respectively. Given enough time (many hours), an external compressive force that exceeds the capillary bed pressure can impair capillary perfusion, leading to ischemia and eventually necrosis and ulceration. Further, for the average person the pressure in the arteries when the heart contracts (i.e., systolic pressure) is 120 mm Hg (1.93 psi). Therefore, a cuff around a limb exerting about 2 psi or more can stop blood flow to a limb. It should be noted that the threshold pressure can be much higher than these pressures; however, consideration must be given to elapsed time where soft tissue breaks down faster with higher pressures. In some embodiments, the pressure modulator is a check valve which modulates the pressure inside the bladder by releasing the gas or fluid inside the bladder once the pressure exceeds a predetermined value, e.g., the threshold pressure. In some embodiments, the limb stabilization device described herein comprises a compressed memory foam which applies pressure to the limb. In some embodiments, the memory foam is pre-compressed to a state which, when released from the pre-compressed state, applies a pressure to the limb not exceeding a predetermined value, e.g., the threshold pressure. As a result, the limb stabilization device described herein surrounds a limb and applies a pressure to the limb at or below a predetermined value, e.g., the threshold pressure. Thus, the limb stabilization device has the advantages of supporting the injured limb in a desirable state to maintain regular blood flow and facilitate healing and recovery without subjecting the limb to detrimental pressures, e.g., pressures exceeding the threshold pressure.

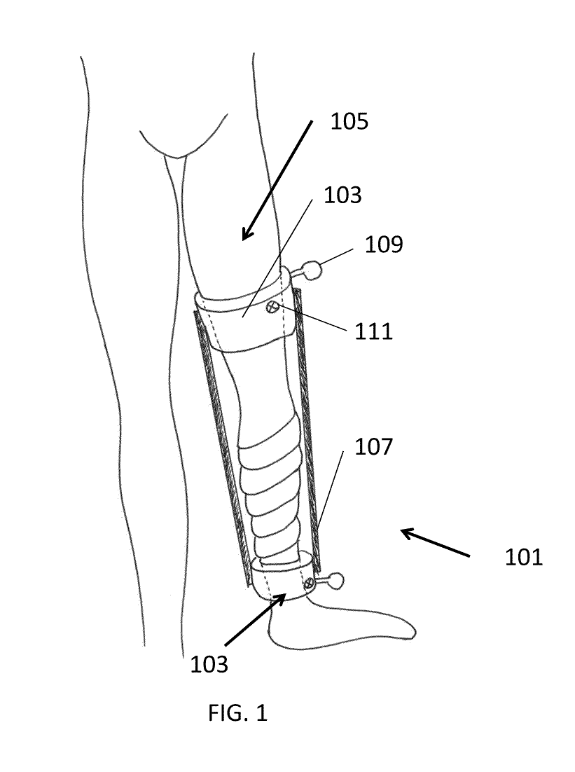

The device presented in FIG. 1 presents one embodiment of a limb stabilization device, which will be referred to as Device 101. Device 101 comprises collars 103 that safely interface with the limb 105 so as not to constrict blood flow and a stiff beam 107 that bridges the collars 103 to reinforce the limb 105. Device 101 may further include a speed inflator 109 (e.g., CO₂ cartridge), and a check valve 111. The check valve is configured to modulate the pressure inside the bladder by releasing the gas or fluid inside the bladder once the pressure exceeds a predetermined value, e.g., 1 psi.

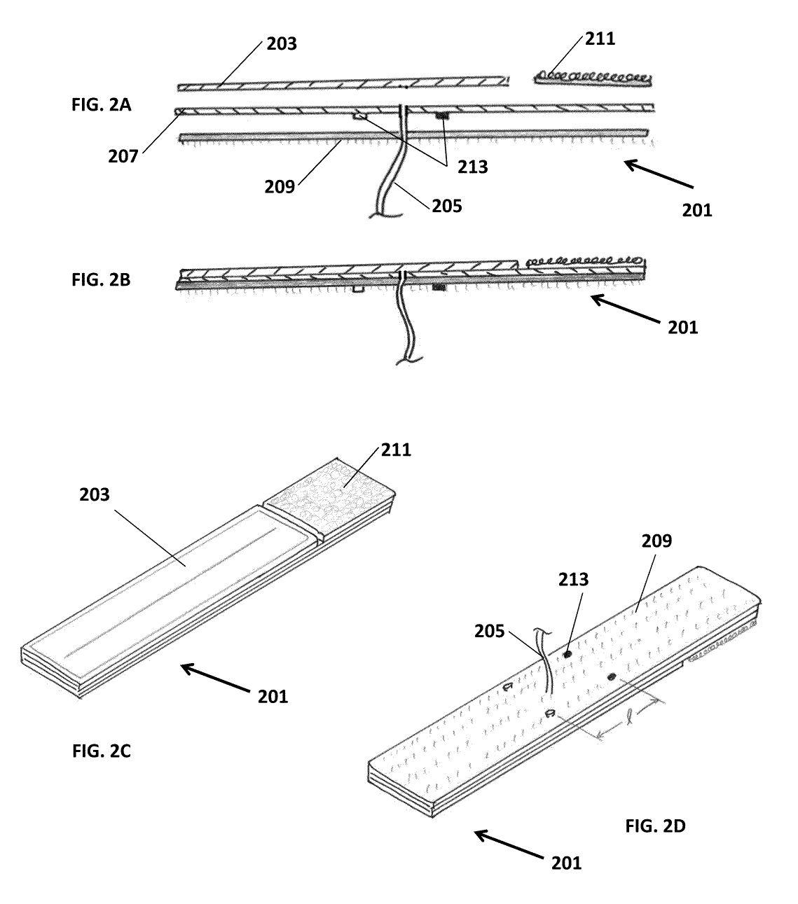

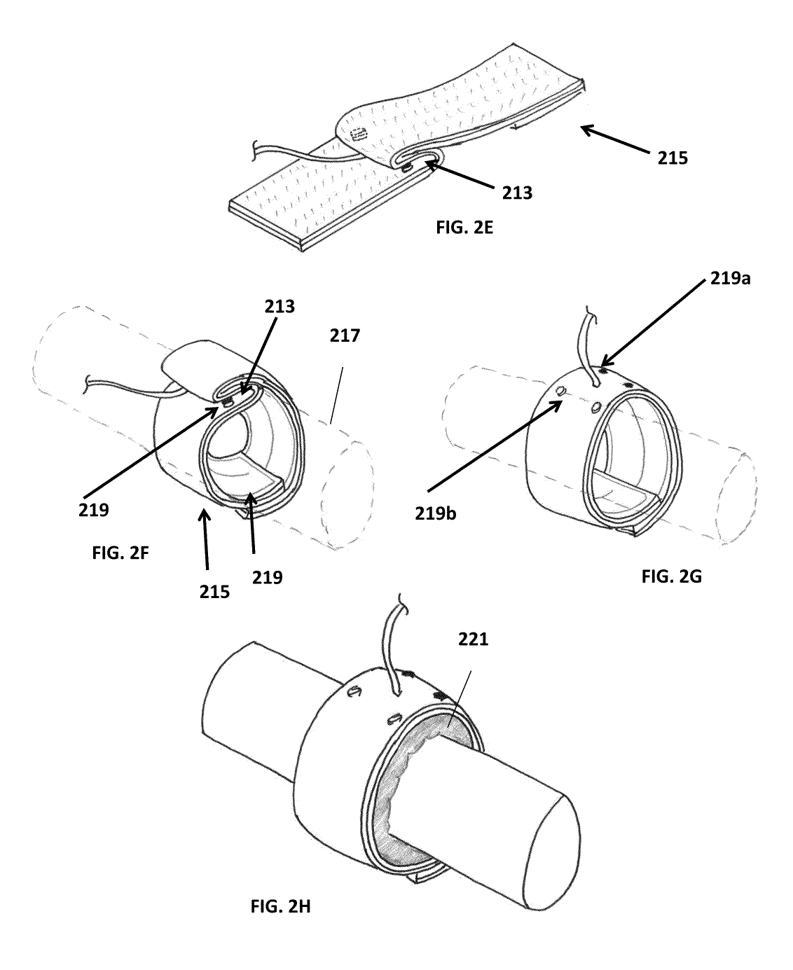

The collars 103 can take on several different forms. FIGS. 2A-H present one exemplary method of construction and attachment of an inflatable collar. FIG. 2A presents a cross-section exploded side view of the inflatable collar 201's components comprising a flexible polyurethane sheet 203 bonded at the perimeter to another flexible polyurethane sheet 207 to form an air tight bladder that can be pressurized with a fluid line 205. The flexible sheet 207, can serve as a connection point for the other components including hook 209 and loop layers 211 and snaps 213. The flexible sheet 207 can also have strain limiting properties to limit radial expansion of the collar. FIG. 2B presents the collapsed cross-section side view of the assembled inflatable collar 201, with FIG. 2C providing an isometric view of the inside surface (skin facing side) of the collar 201, showing loop layers 211 and air bladder 203. FIG. 2D presents an isometric view of the outward facing side of the collar 201, showing the pressurized fluid line 205, hook 209 and snaps 213. In certain embodiments, the bladder can be made from materials such as thermoplastic polyurethane (TPU), TPU coated nylon, vinyl, plastic or any material that can be formed into an inflatable bladder. In certain embodiments, the bladder is made from an elastomer. In other embodiments, the bladder is a plastic bag.

FIGS. 2E-H depict the function of the snaps and deployment of the collar. The snaps or any connection means (e.g., hook and loop, cinch straps, buckles, and break away stitching) are used to create built-in slack in the collar, which is used to maintain consistency of application.

FIG. 2E depicts the snap holding a built-in slack or a fold 213 in the collar 215. During deployment the collar 215 is wrapped around the limb 217, e.g., shows as the dashed cylindrical outline in FIG. 2F. In this embodiment, the hook and loop surface 219 provides the connective means for attaching the collar to the limb. Once the collar 215 is attached, the built-in slack 213 can be released by releasing the snaps 219a and 219b (FIG. 2G). In this way, the thickness of the pressurized bladder that fills the space between the collar 215 and the limb 217 (FIG. 2H) can be controlled or tuned by the length of slack 213 built into the device when the bladder 221 is inflated. As a result, the thickness of the inflated bladder can be fine-tuned to be roughly the same as the limb to be supported, from large circumference limbs to small ones. Thus, the same collar can be used to accommodate limbs of all sizes because the built-in slack and/or the hoops and loops allow the user to set the circumference of the collar based on limb sizes. The slack may also serve to adjust the pressure applied to the limb by the collar.

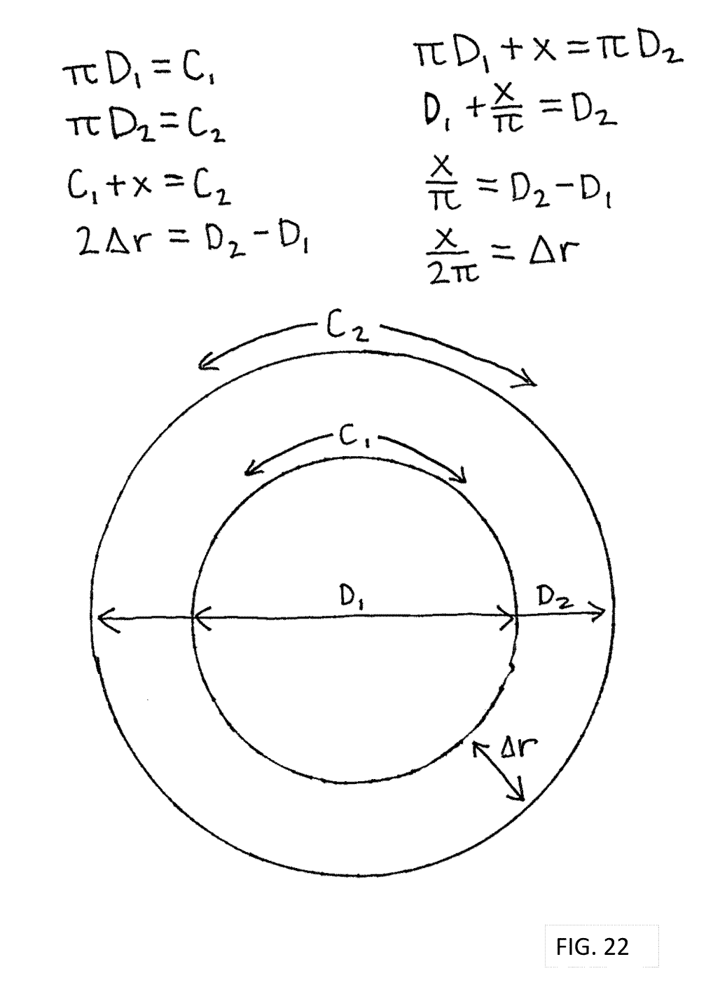

If the built-in slack is too long, the collar will not engage the limb properly and stabilize it even at maximum inflation after the slack is released. On the other hand, if the built-in slack is too short, the collar could apply undesirably high pressure on the limb after the slack is released, which over time (on the order of hours) may cause ulcers and necrotic tissue on the limb. In some embodiments, the desirable length of the built-in slack can be calculated as shown in FIG. 22. The length of the built-in slack x maybe considered as the difference in length between circumferences C₁ and C₂, and Δr is the space between the inside of the collar and surface of the limb. In this case, Δr would be the inflated thickness of the collar from which the maximum built-in slack's length can be calculated. In some embodiments, the desirable length of the slack (x) can be calculated as x/(2π)=Δr. The built-in slack as described herein ensures that the limb is sufficiently supported and stabilized by the limb stabilization device, e.g., via the pressured bladder or the compressed memory foam, while at the same time not being subjected to undesirable high pressure beyond the threshold pressure. The adjustment of the slacks can be made by a user in the field (e.g., a medic) and pre-set by a manufacturer of the device.

Other safety features can be incorporated into the device as well. For example, in certain embodiments, a pressure modulator, e.g., a check valve, can be incorporated into the collars (or other devices described herein) to prevent over pressurization and to accommodate limb swelling. If the collar does not accommodate some limb swelling and/or is over pressurized, it may become a tourniquet. This is another advantage of the built-in slack, as the limb would have to swell considerably (i.e., a large Δr) before reaching the outside diameter of the collar. Furthermore, the amount of built-in slack can be tuned to accommodate the typical amount of limb swelling following a trauma. In some embodiments, regulation of the internal bladder pressure at or below a threshold value (˜1 psi) will prevent the collar (or other devices) from restricting the limb's blood flow.

Any inflation devices or inflation source known in the art can be used to inflate the bladder in any of the devices described here (e.g., devices 1-3 described herein). Non-limiting examples of the inflation device include hand pumps, a chemical reaction, compressed gas (e.g., air or CO₂) cylinders or cartridge, and fluid pumps.

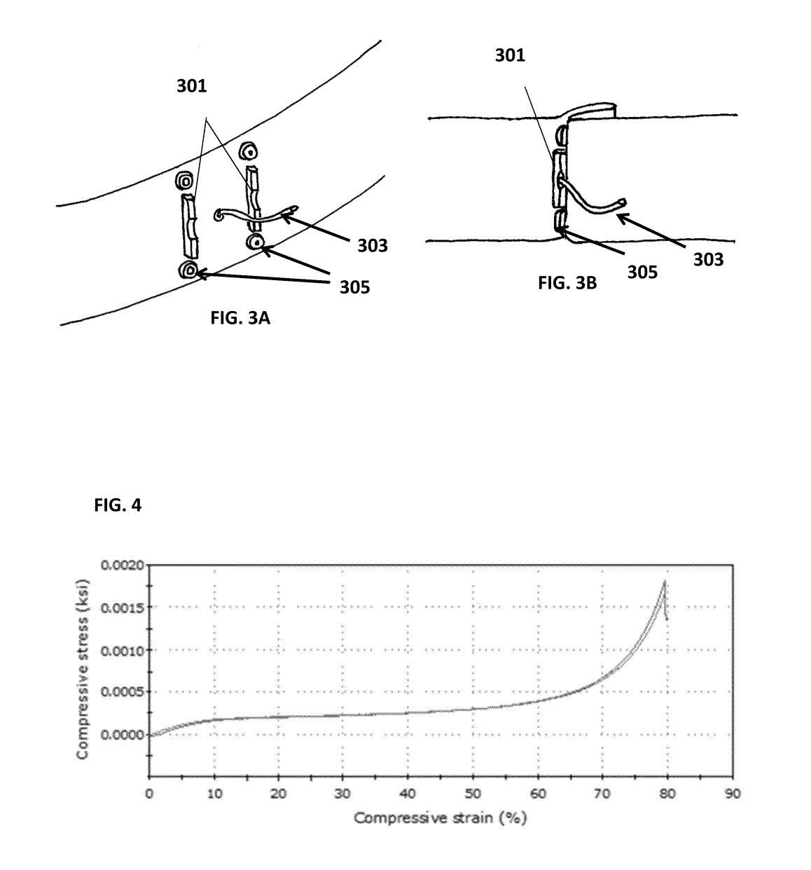

FIGS. 3A-B present another safety feature where a pinch valve 301 closes off the inflation line 303. As shown in FIG. 3A, pinch valve 301 is in the open state when the connective mechanism 305 holding the built-in slack has been released. As shown in FIG. 3B, pinch valve 301 is in the close state because the connective mechanism 305 holding the built-in slack has not been released which prevents collar inflation. This prevents the operator from pressurizing the bladder before the built-in slack has been released. In other embodiments (not shown), a breakaway connection mechanism (e.g., a break away thread) could be triggered by the collar inflation and enable automatic release of the built-in slack.

Another embodiment for preventing over-pressurization by the limb stabilization device is to use foam such as memory foam. These foams have a non-linear stress-strain response that is suitable for applying predictable and safe pressure around a limb. FIG. 4 plots the compressive stress vs. compressive strain of a sample piece of memory foam where the foam exhibits a consistent stress response for up to 50% compression. In this example, the stress does not exceed a threshold pressure, e.g., 1 psi, until about 75% compressive strain. Thus, in some embodiments, the stress of the memory foam is maintained at a level not exerting pressure more than the threshold pressure described herein. In some embodiments, a collar using memory foam could be applied with about 10 to about 15% compressive strain giving the room for the limb to swell without changing the pressure applied to the limb. The compressive strain may be determined by using a standard curve such as the one shown in FIG. 4. In some embodiments, a collar using memory could be applied with about 10%, 15%, 20%, 25%, 30%, 35%, 40%, 45%, 50%, 55%, 60%, or 65% compressive strain or in a range bounded by any two values disclosed herein. Furthermore, memory foam eliminates eliminating pumps or plumbing, leakage rates, and the challenges of managing bladder pressures with changing temperatures or altitudes.

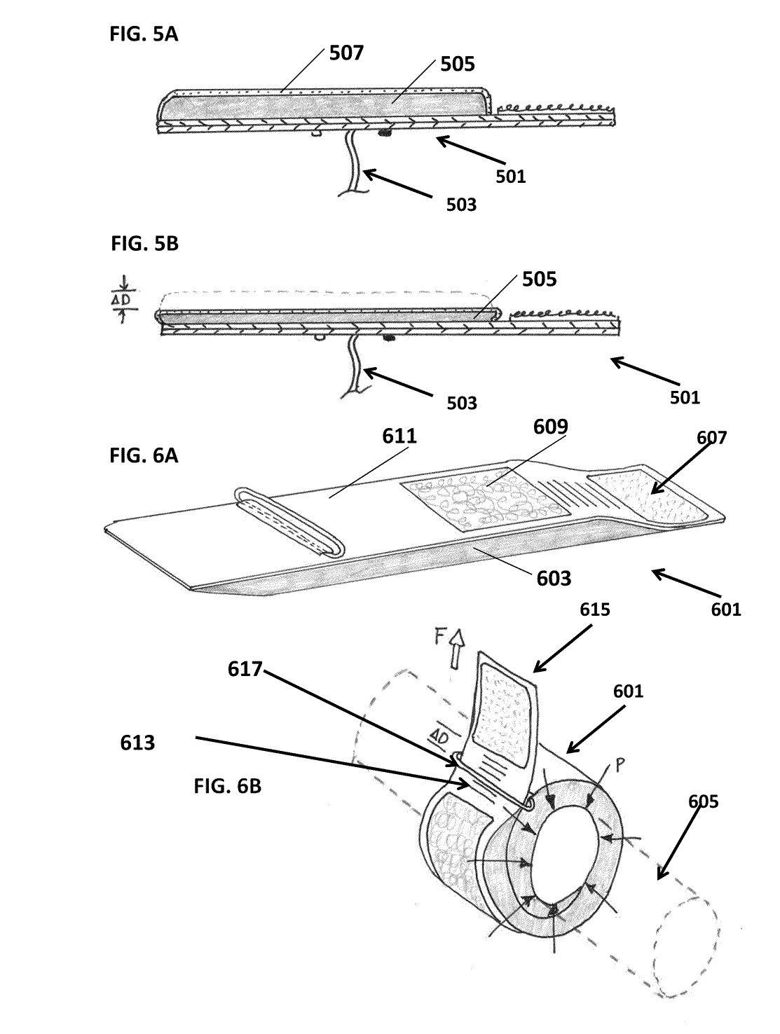

One embodiment of a memory foam collar 501 is presented in FIG. 5A, where the memory foam 505 is inside the bladder 507 and takes the place of the air in the inflatable collar. In this example, the bladder 507 can be evacuated of air via the fluid pressurization line 503 thus causing the memory foam to lie flat (FIG. 5B) (i.e., foam 505 is in a compressed state). In some embodiments, the operator proceeds as described earlier by applying the collar 501 in the compressed state to the limb and releasing the built-in slack. A vacuum seal on the bladder can then be broken to bring the bladder pressure to atmospheric pressure (or the bladder can then be pressurized to cause the collar to quickly conform to the limb and provide the needed stabilization). Over a certain amount of time, the memory foam 505 expands to conform to the limb thereby replacing the role of the pressurized fluid. As another alternative, the bladder could contain a foaming agent (or connection to a foaming agent) such that upon catalyzation and/or foam formation, the foam would fill the bladder and cause the collar to conform to the limb. Such foaming mechanism may be applied to other limb stabilization devices described herein.

In other embodiments, the memory foam 603 does not need to be contained in the bladder and vacuum-sealed, as is illustrated in FIGS. 6A-6B. Referring to FIG. 6A, the collar may include hook 607 and loop 609 for securing the collar around a limb and also a strain limited layer 611. A memory foam lined collar 601 could be applied directly to the injured limb, e.g., limb 605 shown in FIG. 6B. In this example (FIG. 6B), the collar 601 can be applied to the limb 605 and tightened with a force, F (direction as shown in FIG. 6B), to tighten the collar by an amount, ΔD. Markings 613 on the collar tab 615 running through ring 617 can provide an indication of the amount of tightening. The non-linear visco-elastic force properties of the foam would adjust to apply a uniform pressure around the limb at a level that does not to constrict blood flow.

In some embodiments, with respect to integration into the splint device 702, there are several methods for connecting the collars with the stiff beam as depicted earlier in FIG. 1. Any beam securing means can be used with any collar embodiments described herein, and the invention is not limited to the particular beam and collar combination shown in the examples. The stiff beam can made from material such as fiberglass, carbon fiber, hand formable aluminum, and rigidizing foam to name a few. FIG. 7 presents one embodiment where collars 701, 703, and 705 each have a channel, i.e., 701a, 703a, and 705a, respectively, through which the stiff beam 707 can pass. The position of each collar (701, 703 and 705) can be adjusted along the length of the stiff beam 707 (along directions shown by arrow in FIG. 7) to accommodate different limb lengths and wound locations. This also enables easy handling/management of all the collars since they are all attached to the same stiff beam. Furthermore, in some embodiments, for fluid pressurized collars, the stiff strip can serve as a common routing point for fluid pressure connections. Alternatively, the collars can be designed to attach and detach from the stiff beam. For example, the channel can be a fabric constructed with a fastener (e.g., snaps, buckle, hook and loop, etc.) that can be released to separate the collar completely from the stiff strip and can be used to re-attach the beam.

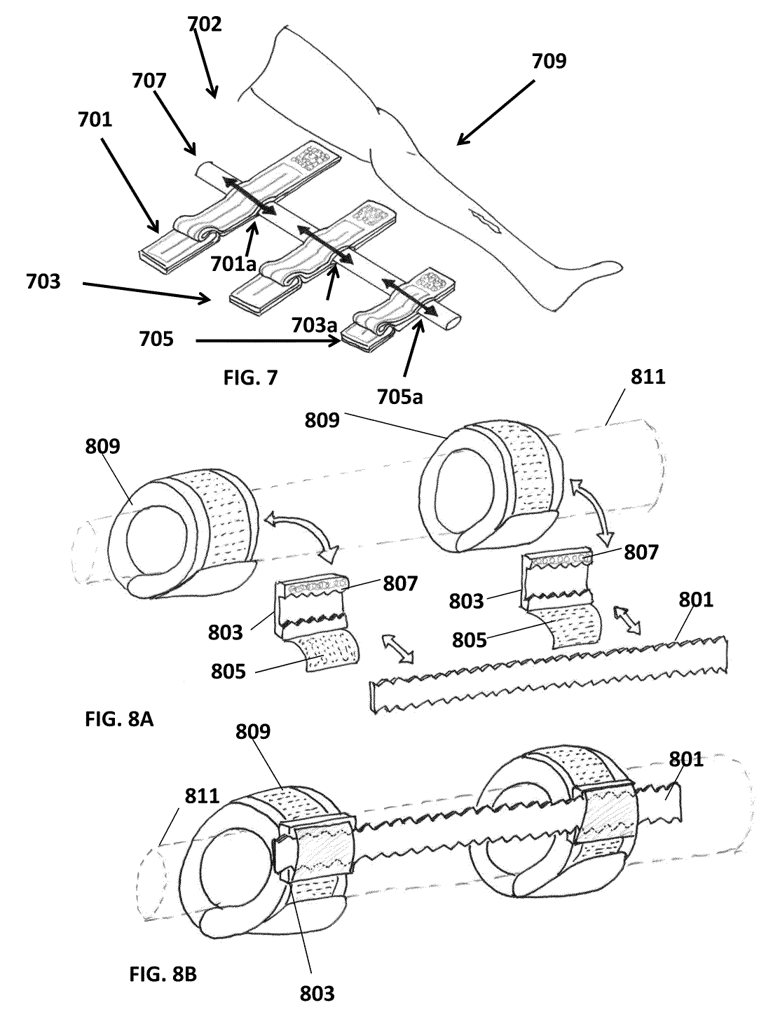

In another embodiment, the collars can have mechanical features that engage and lock onto the stiff beam. For example, FIG. 8A depicts a splinting device for stabilizing limb 811 containing collar 809, a stiff beam 801 with a toothed profile and mounts 803 attached to collar 809 that accepts the tooth profile. In this design, the mount position can be adjusted along the stiff beam and then held in place by a flap of hook material contacting loop 807 to cover the open face of the mount 803 to constrain the stiff beam 801. Any number of connective mechanisms/locking mechanism could be used such as snaps, magnets, buckles and etc. FIG. 8B depicts an assembled view of this construction, showing a device including still beam 801 secured by mounts 803 and collar 809 to stabilize limb 811.

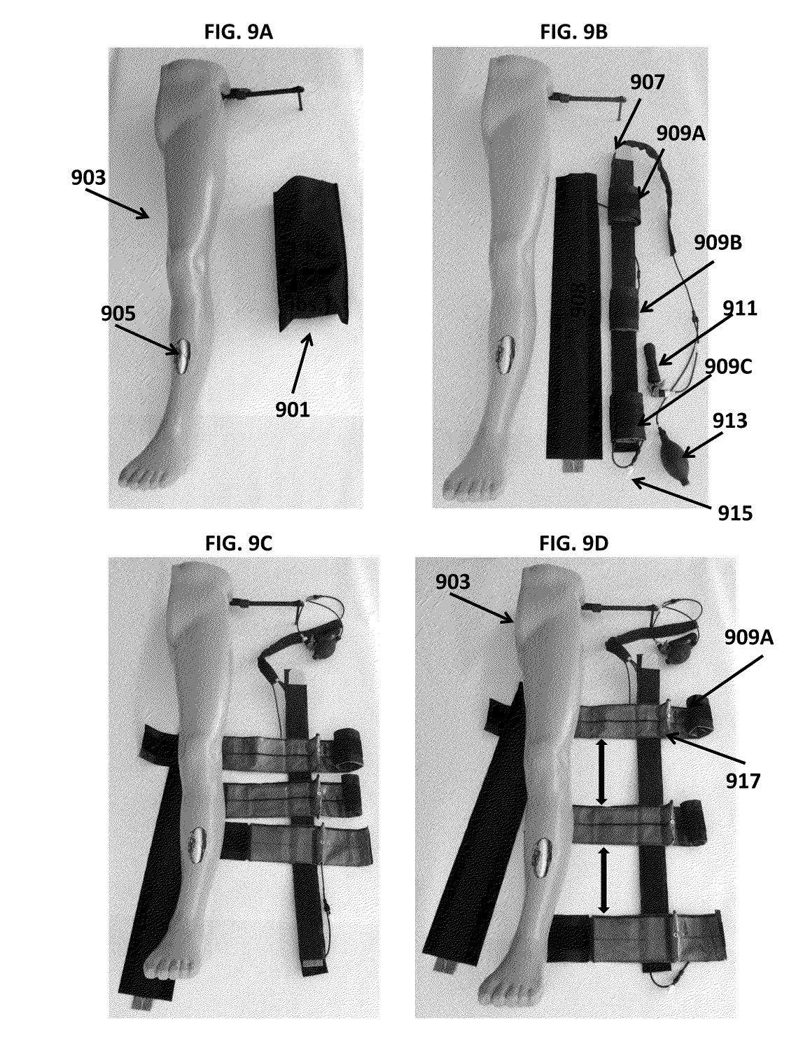

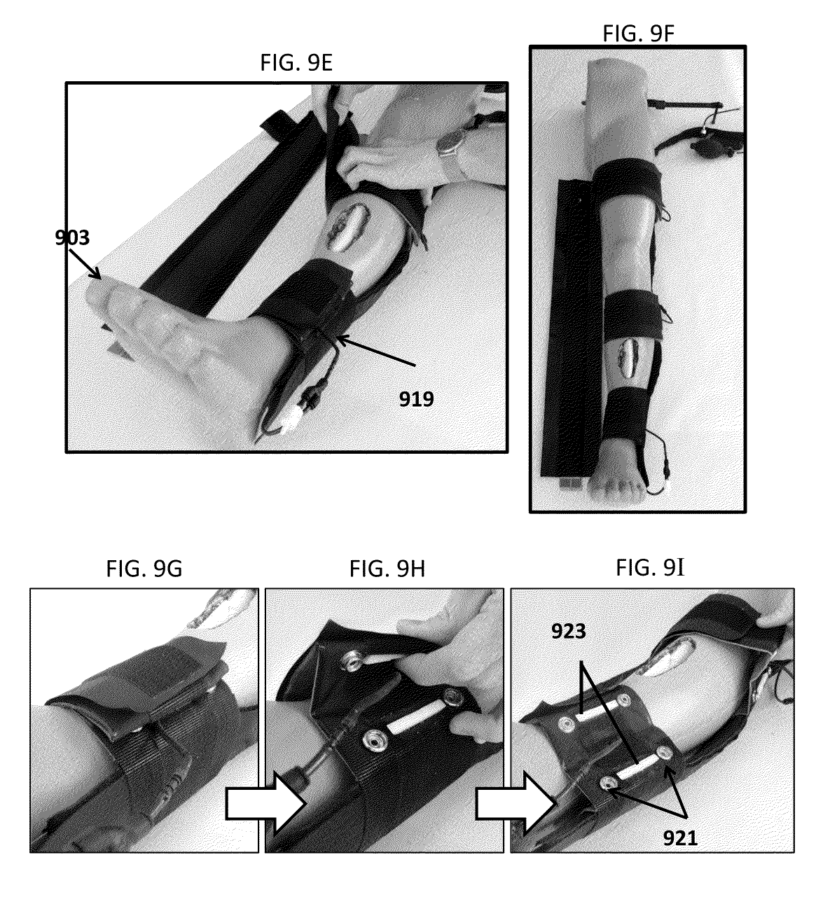

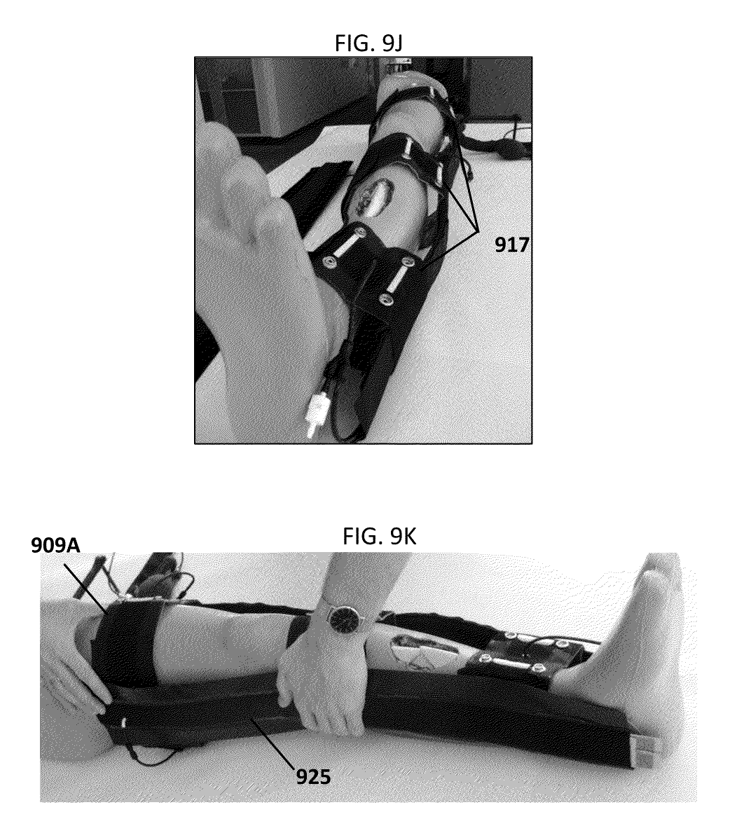

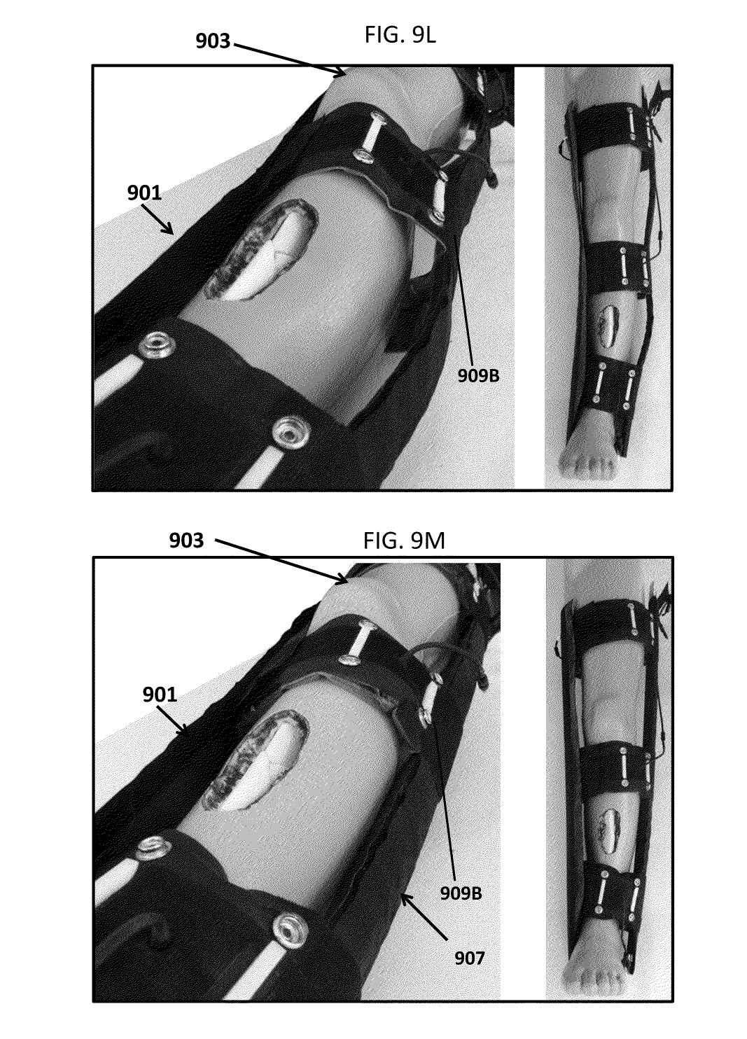

FIGS. 9A-M present one proposed physical embodiment of an inflatable splint according to one or more embodiments. However, it should be noted that the collars and stiff beam may take on other forms as previously described and that elements and features of the splinting device can be used in any combination. FIG. 9A presents the splint 901 in its packaged state adjacent to an injured limb 903 with a grade 3A tib-fib fracture 905. The components are designed to roll and/or fold down into a small pack for easy portability. In FIG. 9B, the splint contents have been removed from the package, which comprise two inflatable rigidizing beams 907 and 908, three inflatable collars 909A, 909B, and 909C, a speed inflator 911 (e.g., CO₂ cartridge), a hand inflator 913, 7 psi check valve (not shown), and a 1 psi check valve 915. The collars are unrolled in FIG. 9C, and their positions are adjusted in FIG. 9D. Note that as shown in FIG. 9D, collar 909A includes a built-in slack 917 to adjust the circumference of the collar conforming to the limb. The collars are attached to the limb 903 via hook and loop surfaces 919 on the collar 909A-C (FIG. 9E-F). Following collar attachment, the built-in slack 917 is released (FIGS. 9G-I) by opening the snaps 921 to release the pinch valves 923. FIG. 9J shows a perspective view of the collars with the built-in slack 917 released. In some embodiments, more than one stiff beam is used for supporting the injured limb. FIG. 9K depicts a hook and loop attachment of a second stiff beam 925 to the collars, e.g., collar 909A. FIG. 9L presents a perspective view and a top view of the device 901 mounted to the leg 903 before pressurization (e.g., collar 909B is unpressurized). FIG. 9M presents a perspective view and a top view of the pressurized device 901 where the inflatable collars (e.g., collar 909B) conform to the limb 903 and serve as anchors for the stiff beam 907 to provide support and alignment to the injured limb 903.

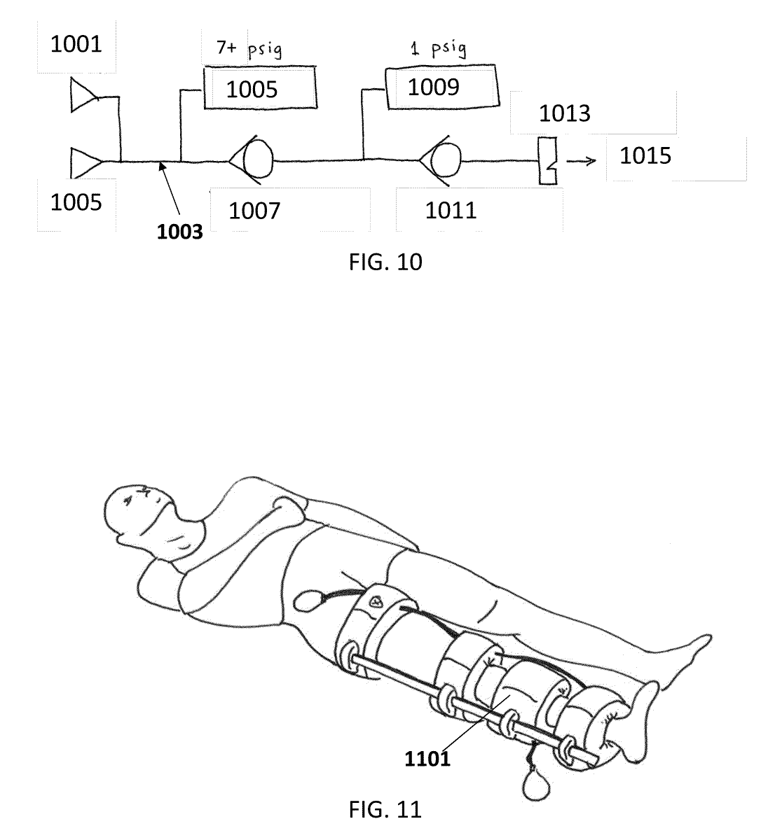

In any embodiments described herein, the rigid beam can be an inflatable rigidizing beam which upon fluid pressurization becomes rigid (see, e.g., beams 907 and 908 in FIG. 9B). In the device described herein, a pressure modulator, e.g., check valve, can be used to regulate the pressure of the inflatable rigidizing beam and/or the collars. In one configuration, the stiff beam and the collars can be on separate fluid pressurization lines. This provides greater control over the sub-systems so that the beam and the collars (or bending actuators and conformal material layers described herein) can have different stiffness. However, it does add additional component parts (e.g., hand pump, valves, etc.). Alternatively, in some embodiments, the plumbing and check valves can be arranged so that components pressurize from a single inflation line in series from highest pressure to lowest pressure (see, e.g., FIG. 10). For example, shown in FIG. 10 is an inflation line 1003 used in a splinting device described herein. A CO₂ inflator 1001 and optionally a hand inflator 1005 are connected to the line 1003. The rigidizing inflatable stiff beam 1005 has a check valve 1007 that limits inflation pressure to about 7 psi, then when this pressure is reached, excess pressure can be used to pressurize the collars 1009 to about 1 psi. When the collars have inflated, the entire device is fully pressurized. Furthermore, the excess pressure from the last check valve 1011 (e.g., 1 psi) in the system can be outfitted with a whistling device 1013 to give the operator an auditory signal that the device has fully inflated and to release the extra gas into the atmosphere 1015.

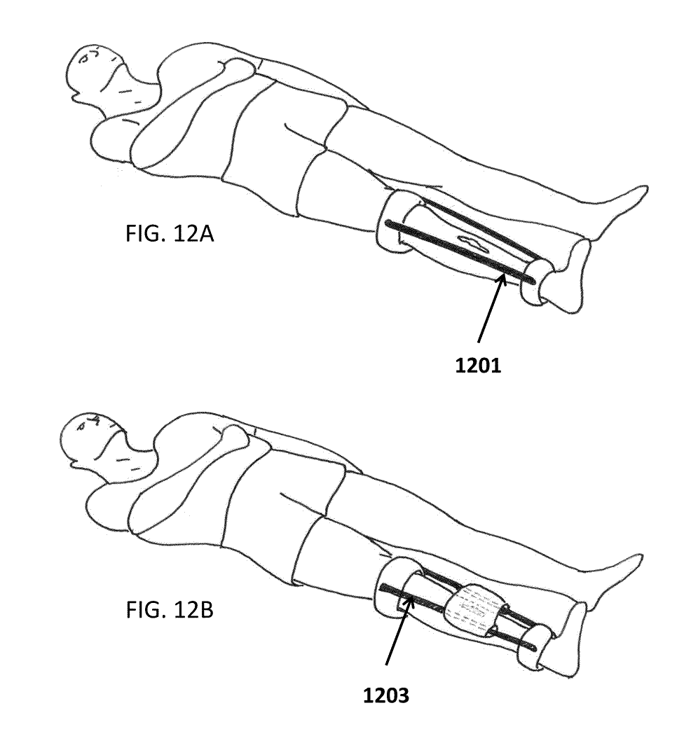

In certain embodiments, the collars can be designed to serve other roles in addition to acting as anchor points. For example, in FIG. 11 a collar 1101 can be added to apply compression to the wound to control bleeding. The collar can also be lined with hydrophilic material to soak up blood. Additionally, a wound compression device does not have to be a collar that wraps around the limb, but instead can anchor to other features on the stabilization device such as the beams. An embodiment of this is depicted in FIG. 12A-B, where beams 1201 and 1203 are attached to the device as described herein.

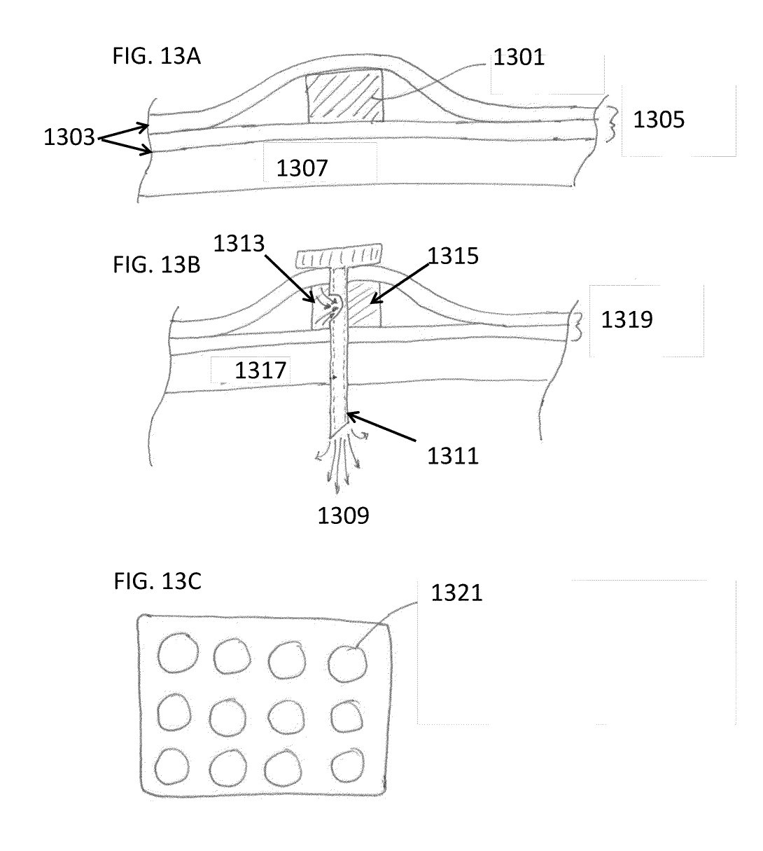

In some embodiments, the collar, wound compression device, or beams can have medicine integrated into the device for rapid delivery, which is important in hostile, and austere environments. For example, a collar can have an inner lining filled with a quick clotting agent to control bleeding or iodine to disinfect the wound site. Furthermore, the collars can contain patches or capsules 1301 (FIG. 13A) for delivering a range of medicinal and therapeutic treatments. Thus, the material layers 1303 made from soft material 1305 and containing medicine capsules 1301 are in contact with the skin 1307 and may be used to deliver medicine to the wound. Thus, in certain embodiments, the medicine can be part of the collar or embedded in the collar/bladder.

The surface of the soft actuator or soft material can offer other functions of such devices for measuring the health of a patient or provide a means of delivering a range of medicinal or therapeutic treatments. For example, FIG. 13B presents one embodiment where a needle 1311 with an opening 1313 on its side can puncture a medicine capsule 1315 in the soft material layers 1319 and channel its contents 1309 into the patient, through skin 1317. FIG. 13C highlights that multiple capsules 1311 each containing the same or different medicines can be arranged to deliver multiple treatments depending on the patient's particular needs.

Traction is also an important factor in limb stabilization and can achieve several different functions. In other embodiments (not shown), a conformal foot covering device may have a ratchet-like interface (e.g., zip tie) with the rigid strip. Any translation of the limb would be locked into position. In another approach, a traction force could be manually applied to the limb, and activation of the limb stabilization device could hold the traction force in place. In yet another embodiment, the stiff strip could provide a large enough linear extending force to apply traction to the limb. In yet another embodiment, the leg and the stiff beam can be manually stretched and then the device activated to hold its new shape. In this manner, the leg and the stiff beam would be stretched at the same time and the stiff strip would be activated to maintain leg traction. It should be noted that in many of these applications minimizing collar slip relative to the patient's skin is important. In one or more embodiments, a skin safe and high friction lining (e.g., FabriFoam) in the collar may be used.

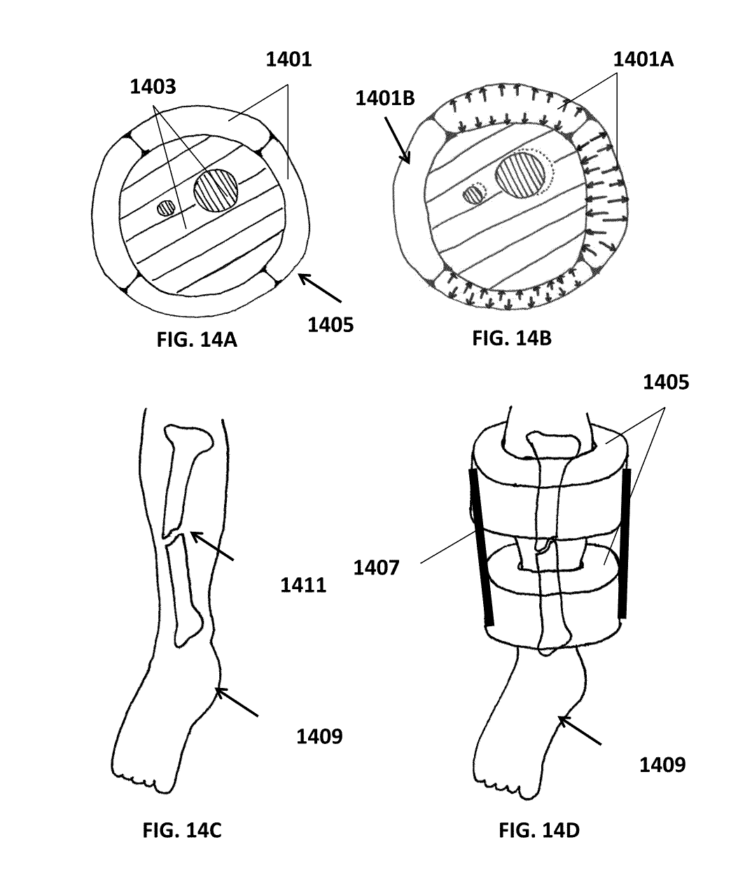

The collars can also include features that are useful at different stages of care. For example, FIG. 14A illustrates a multi-chambered collar 1405 conforming to a limb 1403 and containing four bladders 1401. As shown in FIG. 14B, certain bladders 1401A are selectively pressurized and others such as 1401B are not pressurized. FIG. 14C shows a limb with a broken bone 1411 and FIG. 14D shows that a splinting device containing two multi-chambered collar 1405 and a stiff beam 1407 are used to support this limb 1409. The design of the multi-chambered collar may allow the application of fine-tuned forces to the limb for more control over bone alignment. Thus, in certain embodiments, the bladder 203 can be divided into more than one individual sections and these sections are connected to different fluid sources to selectively pressurize one or more bladder sections.

It should be noted that after the devices disclosed herein have been used and served their purpose, they can be quickly removed by releasing the fasteners and/or deflating the device. Furthermore, many components of the device are constructed from gas impermeable films (e.g. thermoplastic urethanes) and textiles that can be cut with scissors, which offers another method for removal. It should also be noted that other devices can be attached to the splint device for additional functionality. For example, it may be desirable to hold a joint at a certain angle or position while awaiting definitive care, in which case a specialized mechanism could attach to the splint to support the joint orientation. For instance, a foot covering that attaches to the splint could hold the foot in a specific orientation.

In a further aspect, a limb stabilization device is described, including one bending actuator or two or more interdigitating bending actuators configured to surround a limb and apply a pressure to the limb at or below a threshold pressure, wherein the actuator comprises a pressurizable bladder or a compressed memory foam to conform the actuator to the limb. In some embodiments, a pressure modulator configured to regulate the pressure of the bladder to be at or below a threshold pressure can be used. The device may further include a stiff beam to support and stabilize the limb. Alternatively, the bending actuator comprising a pressurized bladder and/or a compressed memory foam provides the required stiffness to support and stabilize the limb.

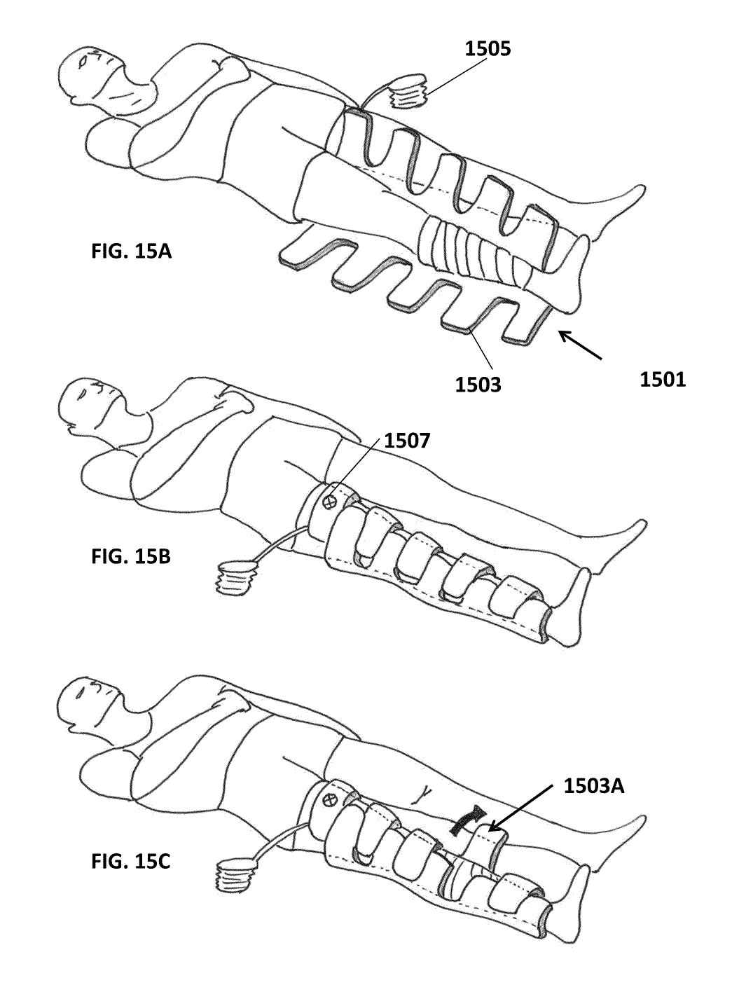

FIGS. 15A-C present an embodiment of the limb stabilization device including one bending actuator or two or more interdigitating bending actuators, which will be referred to as Device 2. This device is a monolithic limb stabilization device where upon fluid pressurization the device conforms around the patient's limb. FIG. 15A depicts the device 1501 containing interdigitating bending actuators 1503 in the deflated stage and positioned under the wounded limb and an inflator 1505. As the device pressurizes, opposing bending actuators (or digits) 1503 come together and interlock, as shown in FIG. 15B. To reinforce the limb, the spine of the device can be made of a stiff material, a rigidizing beam, or can derive stiffness from pressurization (i.e., a higher pressure section). The digits extending from the spine are designed to conform around the limb without applying excessive squeezing forces. A check valve 1507 may be included to ensure the pressure applied does not exceed a predetermined value. In some embodiments, these digits can be soft bending actuators, which has a desirable feature in this application in that they are back drivable. This enables medical personnel to peel back the actuator (if necessary) to inspect the wound site and upon release, the actuator would return to its original position (FIG. 15C, showings one soft bending actuator 1503A is peel back). In other embodiments, the device comprises a single bending actuator which upon pressure (pressured bladder or released compressed memory foam) may wrap around and conform to the limb and apply pressure to stabilize the limb. Furthermore, Device 2 can be removed by deflating the device or it can be cut off.

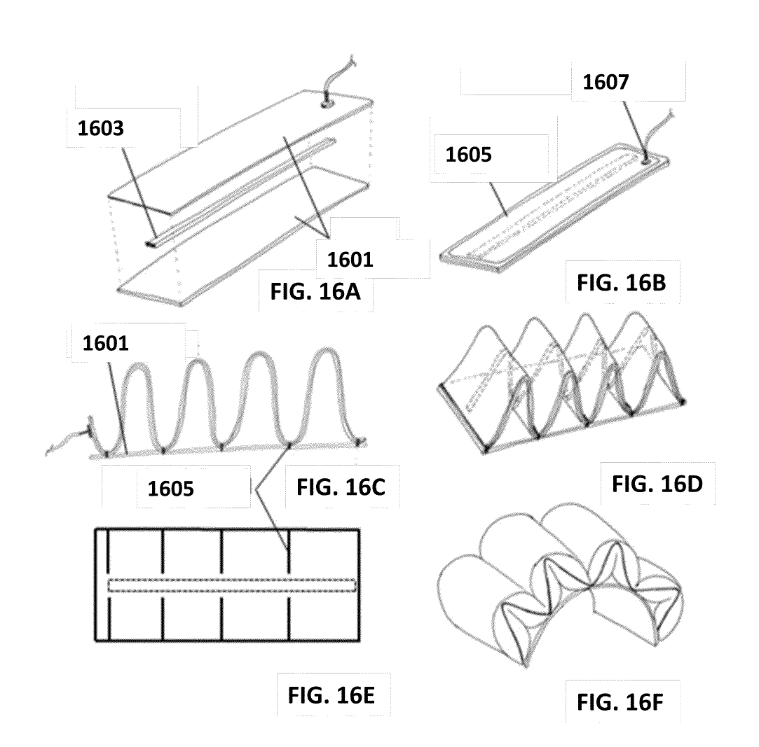

There are several methods for creating the bending actuators presented in Device 2. In one embodiment, a soft bending actuator can be constructed from two plastic sheets 1601 that are thermally bonded along their perimeter 1605 and enclose an open cell foam strip 1603 (FIGS. 16A-B) with a pneumatic connection at line 1607. The bladder can then be configured into a serpentine shape (FIG. 16C offers a side view, and FIG. 16D presents an isometric view), and certain sections (FIG. 16E) are bonded to another plastic sheet. When inflated, each period (i.e., the spacing of the serpentine segments) of the serpentine bladder inflates against its neighbor producing a bending motion (FIG. 16F). Furthermore, the amplitude (i.e., height of the serpentine profile), period, depth and length of the device can all be adjusted to tune the bending force and range of motion of the device. It should be noted that the open cell foam keeps the entire length of the bladder open because serpentine bladder configurations tend to pinch off sections during inflation, which produces non-uniform actuation.

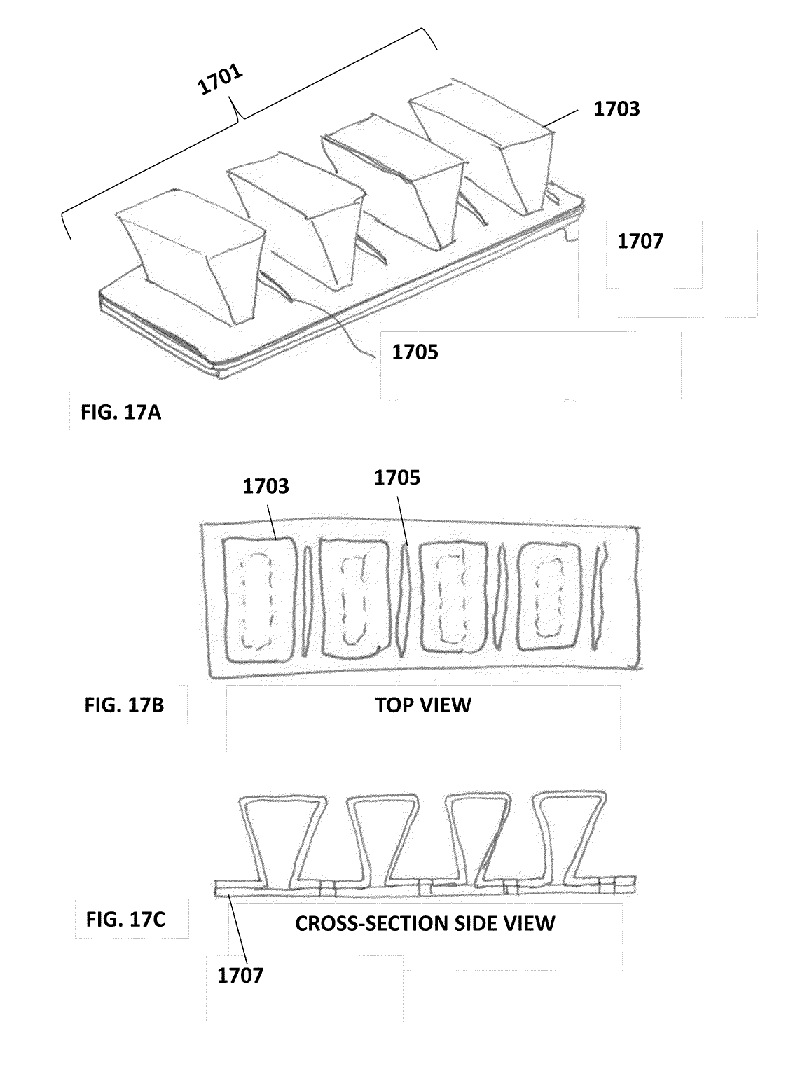

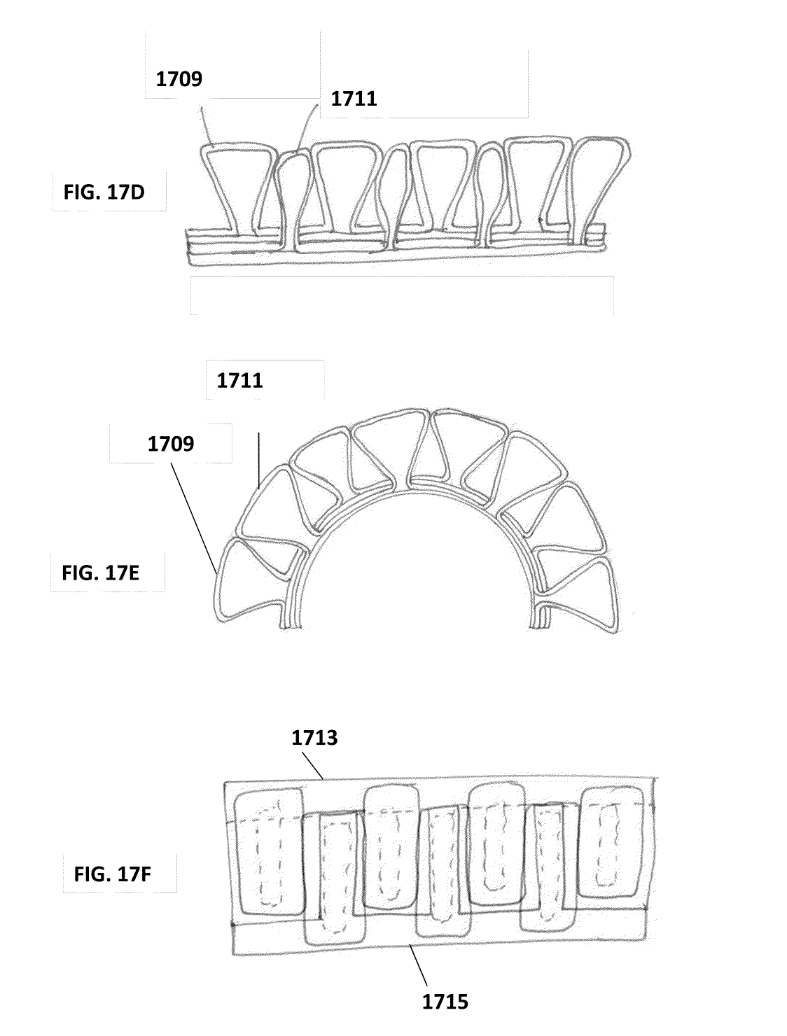

A soft bending actuator can also be constructed by thermal forming the desired inflated shape of the bellow where a row of bellows is thermal formed and bonded to a strain-limiting layer. When the bellows are inflated, they will inflate into their neighbors causing the structure to bend about the strain-limited layer. This design has a limitation in that it is hard to thermal form a high density of bellows. One approach is to interweave two actuators. FIGS. 17A-C present different views of a bellow actuator 1701 with openings 1705 between each of the bellows 1703. In this design one bellow actuator can be interwoven with another by passing the bellows through the openings 1705 (FIGS. 17A and B) on the strain limited bottom layer 1707. This increases the number of bellows per unit length. FIG. 17D illustrates a cross-section side view of two interwoven actuators 1709 and 1711 in a deactivated state (i.e., no pressure), and FIG. 17E illustrates the same two interwoven actuators 1709 and 1711 in the activated state (i.e., pressurized). FIG. 17F illustrates a top view of another approach where two bellow actuators 1713 and 1715 can have a tab or tooth pattern that are interwoven. Another approach (not illustrated) is to insert material to occupy the space between neighboring bellows (i.e., the material could be rigid foam).

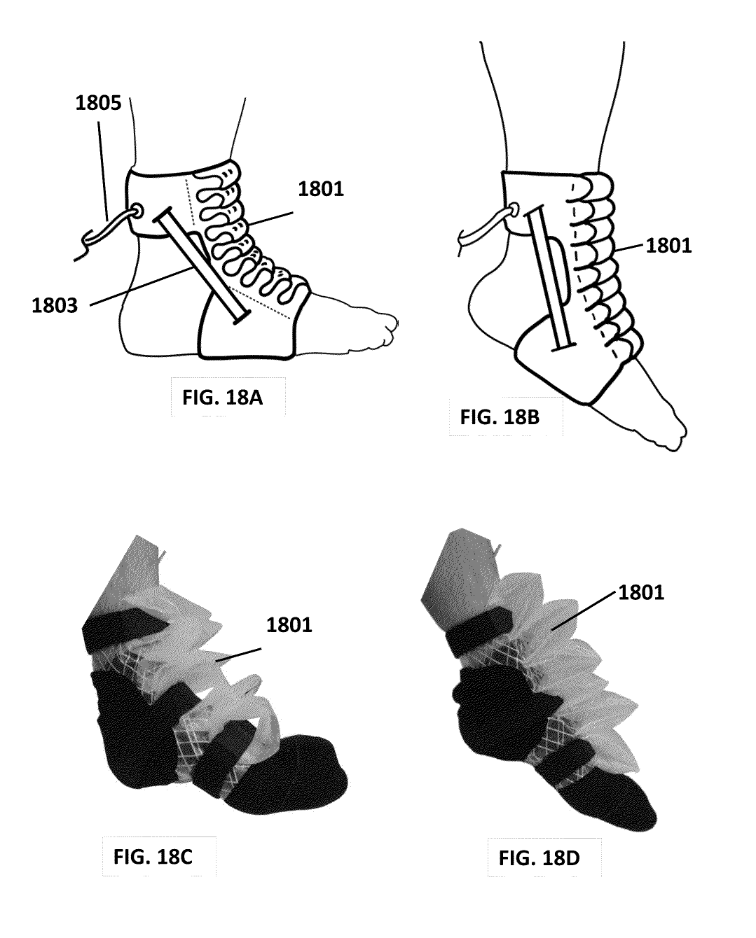

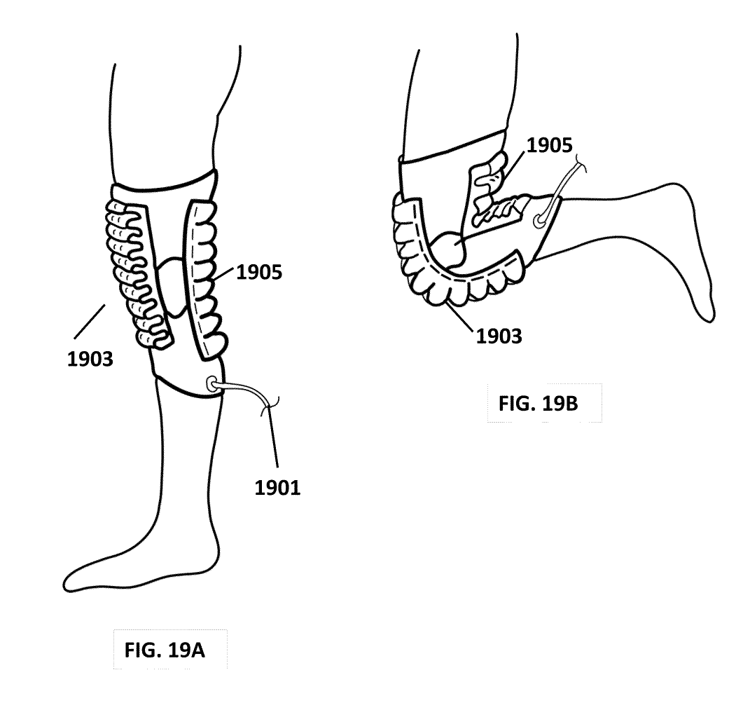

In some embodiments, the actuators and devices described herein are used in fields beyond limb stabilization. For example, these soft bending actuators can be applied to joints such as the ankle, knee, elbow and so forth to provide assistive torques or provide continuous passive motion to joints for patients recovering from surgery. FIGS. 18A-B present one embodiment of the device applied to the ankle where the actuator supports plantar flexion and an elastic band 1803 supports dorsiflexion. The device has a pneumatic line 1805 for pressurization fluid. FIGS. 18A and 18B show the bending actuator in a relaxed and activated state, respectively. The actuators could also be arranged in an antagonistic arrangement for active plantar flexion and dorsiflexion. FIGS. 18C-D present a physical embodiment of the bending actuator applied to the ankle, showing the bending actuator in a relaxed and activated state, respectively. An example of antagonistic arrangement is presented in FIGS. 19A-B, where it is the bending actuators in support knee flexion and extension. Specifically, in FIG. 19A, the device has a relaxed bending actuator 1903 at the front keen and an activated bending actuator 1905 on the back side of the knee. The leg is stretched straight in FIG. 19A. In FIG. 19B, the knee can be bent so that the bending actuator 1903 at the front keen is activated and the bending actuator 1905 on the back side of the knee is relaxed. The device has a pneumatic line 1901 for pressurization fluid.

In a still further aspect, a limb stabilization device is described, including a conformal material layer configured to wrap around a limb and apply a pressure to the limb at or below a threshold pressure, wherein the conformal material layer comprises a pressurizable bladder and/or a compressed memory foam; a pressure modulator configured to regulate the pressure of the bladder to be at or below a threshold pressure; and optionally at least one beam connected to the surface of the conformal material to support the limb. The conformal material layer may be wrapped around the injured limb before the bladder is pressurized or the compressed memory foam is released.

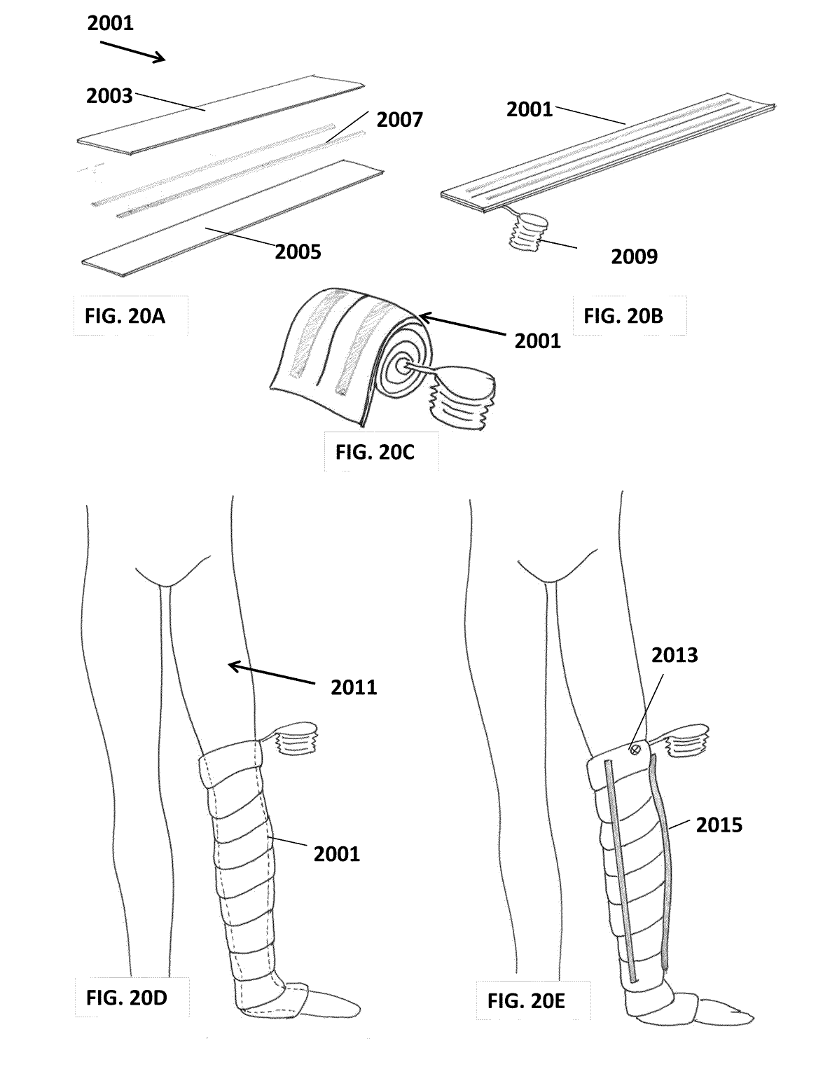

FIGS. 20A-E present an embodiment of the limb stabilization device including a conformal material layer configured to wrap around a limb, which will be referred to as Device 3. This device 2001 comprises a conformal material layer having the form factor of a traditional bandage with a bladder that runs the length of the bandage. FIG. 20A presents an exploded view of the device 2001 which comprises two material layers 2003 and 2005 that are sealed together to form a bladder with foam strips 2007 on the interior to prevent kinking of any air channels. FIG. 20B presents the assembled view of the inflatable bandage device, which contains a flat pack operated pump 2009. FIG. 20C demonstrates that the device 2001 can be rolled similar to a traditional bandage. In its inflated state, device 2001 wraps around the limb 2011 (FIG. 20D). In FIG. 20E, the device further includes one or more check valves or pressure relief buttons to release excess pressure and one or more stiff strips 2015 reversibly adhere to bandage and support alignment.

Device 3 creates a cushion of air around the injured limb. This gives the limb considerable volume in which to swell. Furthermore, stiff strips can be attached (e.g., via hook and loop, adhesives, glue, buckles, etc.) to the outside of the bandage to reinforce the injured limb. The bandage material can also incorporate novel features such as puncture resistance, self-healing (e.g., medicine could be injected through the bandage layers puncturing or having to remove the bandage), and transparency to inspect the wound without having to remove the bandage. Furthermore, the bandage could also be used to deliver hot or cold therapy.

It should be noted that the inflatable bandage could use memory foam instead of a pressurizable bladder to conform to the limb. Similar to the discussion of the collar design, the memory foam bandage could be contained in a vacuum-sealed bladder where the sealed is released after the bandage has been applied to create a conforming bandage that can accommodate limb swelling. Alternatively, the memory foam can be exposed and held in the compressed state by a stretchable structural element or a stain limited element. Thus, the memory foam can be exposed and simply line the bandage (e.g., the bandage can be stretchable or strain limited).

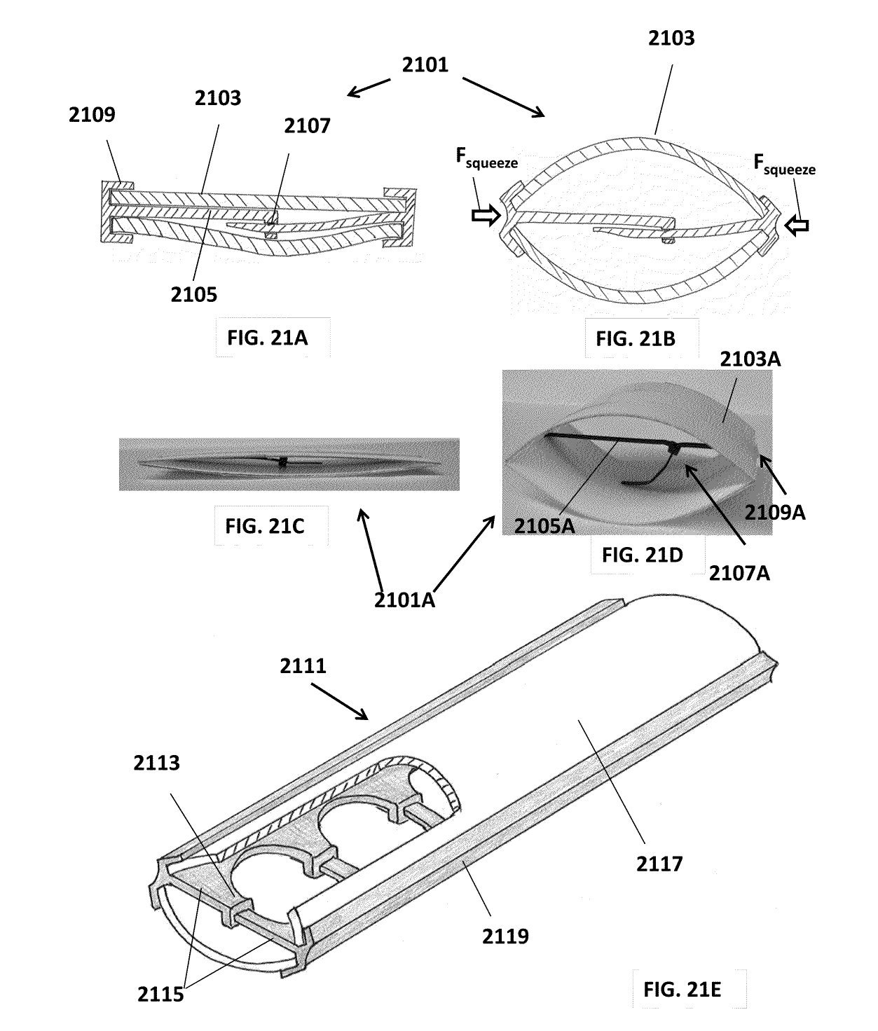

The stiff beam supporting the injured limb in the above proposed devices can take on many different forms including a fixed length tube/rod, a telescoping tube or an unfolding tube. The preferred embodiment is a stiff beam that can collapse to a small form factor (e.g., rolled or folded) for portability and then expand and provide enough stiffness to support the injured limb. FIGS. 21A-D present a rigidizing beam construct that increases the second moment of area to change the stiffness of the structure. In its collapsed state (FIG. 21A), the beam 2101 comprises flexible material layers 2103, a cable 2105 that spans the width of the flexible material layers 2013, a locking mechanism 2107 that connects the cable ends, and a flexible material connector 2109 for holding the flexible material layers in place. When a squeezing force is applied, the flexible material layers 2103 curve, thereby increasing the second moment of area and the stiffness of the beam 2101 (FIG. 21B). This also increases the overlap between the cable ends which is held in place by the locking mechanism. When the squeezing force is released, the locking mechanism maintains the cable overlap thereby putting the cable in tension and storing strain energy in the flexible material layers. FIGS. 21C-D present a prototype beam 2101A in relaxed and activated states, respectively. The beam 2101A contains a nylon zip-tie 2107A as the locking mechanism to lock the cable 2105A, thin plastic layer 2103A for the flexible material layers, and tape 2109A circumferentially wrap around the flexible material layers 2103A as the connector.

FIG. 21E presents a proposed embodiment for a rigidizing beam 2111 where cables and locking mechanisms run the length of the beam. As shown in FIG. 21E, locking mechanism 2113 is configured to lock cables 2115. Flexible layers 2117 are joined at the edge by flexible material connectors 2119. This arrangement gives the operator the ability to cut the beam to length without affecting the structural integrity of the beam, which is a feature not found in rigidizing beams that rely on pressurized fluids. In this embodiment, the operator may have to squeeze at multiple points down the length of the beam in order to increase the stiffness. One advantage of this approach is the operator can control which sections of the beam to stiffen. Another feature that can be incorporated into this design is a fixed length cable that runs transverse to the first, and limits the maximum separation (e.g. curvature) of the flexible material layers. Furthermore, mechanical stops can be integrated into the locking mechanism to also limit the separation of the material layers. These separation limiting features are important for device operation as they can be used to prevent the operator from applying forces that exceed the yield strength of the flexible material layers.

In some other embodiments, the beam is rigidized without physically applying a squeezing force to the structure. An inflatable bladder between the flexible material layers can be inflated to curve the layers and engage the locking mechanisms. In this way the operator can rigidize the beam from a single source. If the bladder leaks or is damaged, the cable and locking mechanism would maintain beam stiffness.

In some embodiments, the rigidizing beam has two states: a first non-rigid or less rigid state and second rigid or rigidized state which is more rigid than the first state. In some embodiments, the rigidizing beam surrounds a bladder. In other embodiments, the rigidizing beam is adhered to opposing surfaces of a bladder. The bladder may be inflated or pressurized by gas, fluid, or any other pressurizing means known in the art. As a result, the pressure inside the bladder is greater than the pressure outside the bladder and the rigidizing beam surrounding the bladder will change shape, e.g., curve, to accommodate the pressure or increase the separation distance between layers. Consequently, the stiffness of the rigidizing beam is greatly increased. This change of stiffness of the beam may be referred to as rigidizing. The rigidizing beam can be used for structural support in applications such as splinting, structural component, construction, or packaging, due to their greatly increased stiffness.

In some embodiments, the rigidizing beam is made of a material which, when curved, results in an increased stiffness. Non-limiting examples of the material include metal, fiberglass, paper, composite wood, and plastic. In some embodiments, the laminate layer is thin and has a thickness of less than 10 cm, 5 cm, 4 cm, 3 cm, 2 cm, 1 cm, 500 μm, 400 μm, 300 μm, 200 μm, 100 μm, 50 μm, 10 μm, 1 μm, 500 nm, 400 nm, 300 nm, 200 nm, or 100 nm, or in the range of 100 nm to 10 cm, or any other range bounded by any of the values noted here. The increased stiffness of the rigidizing beam may be a combination of the pressure inside the bladder and the increased stiffness of the rigidizing beam due to its shape change, e.g., curving or grouping of beams. In certain embodiments, the increased stiffness of rigidizing beam is predominantly a result of the shape change of the beam. In certain embodiments, the stiffness increase of the rigidizing beam due to curving contributes to more than about 99%, 95%, 90%, 80%, 70%, 60%, or 50% of the rigidity of the rigidizing beam after it is rigidized. In these embodiments, the pressure increase inside the bladder does not make a significant contribution to the rigidity of the rigidizing beam.

In some embodiments, the stiffness of the rigidizing beam can be further increased by having multiple laminate layers. In some embodiments, on each side of the rigidizing beam there are more than 2, 3, 4, 5, 6, 7, 8, 9, 10, 15, 20, 25, 30, 40, 50, or 100 layers, or in the range of 2 to 100 layers, or any other range bounded by any of the values noted here. Further examples of the rigidizing beams are described in U.S. application Ser. No. 14/688,210, filed Apr. 16, 2015, the content of which is expressly incorporated by reference herein.

It should be noted that the flexible material layers can be made up of multiple layers of material that are connected (e.g., glued, riveted, ect, together) or are allowed to slide past one another with textured or untextured interfaces.

It should also be noted that in all the devices described herein, fluid pressurization can be achieved by several methods including manually pumping (e.g., squeeze bulb or foot pump), compressed gas cartridge (e.g., CO₂ cartridge), chemical reaction, and electric pump.

In yet another aspect, a method of stabilizing an injured limb using a limb stabilizing device according to any of the embodiments described herein is disclosed. The limb is supported and stabilized by the device while it is treated and healed.

The foregoing and other features and advantages of various aspects of the invention(s) will be apparent from the following, more-particular description of various concepts and specific embodiments within the broader bounds of the invention(s). Various aspects of the subject matter introduced above and discussed in greater detail below may be implemented in any of numerous ways, as the subject matter is not limited to any particular manner of implementation. Examples of specific implementations and applications are provided primarily for illustrative purposes.

While for purposes of illustration a preferred embodiments of this invention has been shown and described, other forms thereof will become apparent to those skilled in the art upon reference to this disclosure and, therefore, it should be understood that any such departures from the specific embodiment shown and described are intended to fall within the spirit and scope of this invention.

What is claimed:

1. A limb stabilization device comprising:
two or more collars configured to apply a pressure to a limb at or below a threshold pressure, wherein the collar comprises a pressurizable bladder and/or a compressed memory foam to conform the collar to the limb; and
at least one beam connecting the two or more collars to support the limb.

2. The limb stabilization device of claim 1, further comprising a pressure modulator for regulating the pressure of the bladder to be at or below the threshold pressure.

3. The limb stabilization device of claim 2, wherein the pressure modulator is a check valve.

4. The limb stabilization device of claim 1, wherein the bladder is in fluidic connection with a gas or fluid pressurization source.

5. The limb stabilization device of claim 1, wherein the gas or fluid pressurization source is a gas or fluid hand pump, a compressed fluid or gas cartridge, or a fluid or gas source generated by a chemical reaction.

6. The limb stabilization device of claim 1, wherein the threshold pressure is less than or equal to 1 psi.

7. The limb stabilization device of claim 1, wherein the collar has a built-in slack to result in an adjustable circumference of the collar based on the size of the limb.

8. The limb stabilization device of claim 7, wherein the built-in slack is released after the collar has been applied to the limb.

9. The limb stabilization device of claim 7, wherein the built-in slack is created by a connection means connecting two non-adjacent portions of the collar.

10. The limb stabilization device of claim 7, further comprising a pinch valve openable when the built-in slack is released.

11. The limb stabilization device of claim 1, wherein the collar comprises a channel to receive the beam.

12. The limb stabilization device of claim 1, wherein the collar is connected to the beam via hook and loop or the collar is mounted onto the beam via one more optionally detachable mount.

13. The limb stabilization device of claim 1, wherein the collar is positioned to compress a wound on the limb.

14. The limb stabilization device of claim 1, wherein the collar is positionable along the length of the beam.

15. The limb stabilization device of claim 1, wherein the beam is an inflatable rigidizing beam.

16. The limb stabilization device of claim 15, wherein the collars and beam are connected to the same or different fluid pressurization source.

17. The limb stabilization device of claim 16, wherein the fluid pressurization source is a hand pump or a compressed fluid or gas cartridge.

18. The limb stabilization device of claim 15, further comprising a check valve for controlling the degree of inflation and internal pressure of the beam.

19. The limb stabilization device of claim 15, further comprising a locking mechanism to maintain the beam in a stiff state.

20. The limb stabilization device of claim 1, wherein the stiffness of the beam is adjustable by a squeezing force applied to the beam.

21. The limb stabilization device of claim 1, wherein the beam and/or collars are made from one or more material capable of being cut, rolled, and/or folded.

22. The limb stabilization device of claim 1, where the memory foam comprises more than one layer of foam, more than one type of foam, or a combination thereof.

23. The limb stabilization device of claim 1, wherein the memory foam is vacuum sealed in the bladder.

24. The limb stabilization device of claim 23, wherein the memory foam is maintained in a compressed state by the vacuum seal and the vacuum seal is releasable after the collar is applied to the limb.

25. The limb stabilization device of claim 1, wherein the memory foam is exposed to the environment and held in a compressed state by a stretchable structural element or a stain limiting element.

26. The limb stabilization device of claim 1, wherein the collar has a high coefficient of friction with skin, is breathable, comprises one or more blood-clotting materials, and/or is fluid-absorbent.

27. The limb stabilization device of claim 1, further comprising a medicine integrated in the collar and/or the beam.

28. The limb stabilization device of claim 27, wherein the collar and/or beam comprises one or more capsules containing the medicine and puncturable for delivery into the patient.

29. The limb stabilization device of claim 1, wherein the collar comprises more than one independently controlled bladder.

30. The limb stabilization device of claim 1, wherein the collar comprises a foam liner.

31. A limb stabilization device comprising:
two or more interdigitating bending actuators each comprising a pressurizable bladder and/or a compressed memory foam and configured to bend along a first direction towards the limb in an interdigitated manner upon actuation to apply a pressure to the limb at or below a threshold pressure, wherein the interdigitating bending actuator is actuatable by bladder pressurization or decompression of the memory foam.

32. The limb stabilization device of claim 31, further comprising a pressure modulator for regulating the pressure of the bladder to be at or below a threshold pressure.

33. The limb stabilization device of claim 31, wherein the limb stabilization device further comprises a beam and the interdigitating bending actuators are connected to the beam.

34. The limb stabilization device of claim 31, wherein the limb stabilization device is made from one or more materials capable of being rolled or folded in the unpressurized state.

35. The limb stabilization device of claim 31, wherein the two or more interdigitating bending actuators wrap around the limb upon actuation.

36. The limb stabilization device of claim 31, wherein the threshold pressure is equal to or below 1 psi.

37. The limb stabilization device of claim 31, wherein after actuation, at least one of the interdigitating bending actuator is bendable along a second direction away from the limb.

38. The limb stabilization device of claim 31, wherein the bladder is in fluidic communication with a hand pump or a pressurized fluid or gas cartridge.

39. The limb stabilization device of claim 31, wherein the interdigitating bending actuator comprises a memory foam liner.

40. The limb stabilization device of claim 31, wherein the interdigitating bending actuator has a high coefficient of friction with skin, is breathable, comprises one or more blood-clotting materials, and/or is fluid-absorbent.

41. A limb stabilization device comprising:
a bending actuator comprising a plurality of sequentially-disposed pressurizable bladders each in fluidic communication with a fluid pressurization source or enclosing a compressed memory foam; wherein upon actuation, the adjacent bladders expand against each other so that the bending actuator bends along a first direction towards the limb to apply a pressure to the limb at or below a threshold pressure; wherein the bending actuator is actuatable by bladder pressurization or decompression of the memory foam.

42. The limb stabilization device of claim 41, further comprising a pressure modulator for regulating the pressure of the bladder to be at or below a threshold pressure.

43. The limb stabilization device of claim 41, wherein the limb stabilization device further comprises a beam and the bending actuator is connected to the beam.

44. The limb stabilization device of claim 41, wherein the bending actuator is a bellow bending actuator.

45. The limb stabilization device of claim 41, wherein the limb stabilization device is made from one or more materials capable of being rolled or folded in the unpressurized state.

46. The limb stabilization device of claim 41, wherein the limb stabilization device wraps around the limb upon actuation.

47. The limb stabilization device of claim 41, wherein the threshold pressure is equal to or below 1 psi.

48. The limb stabilization device of claim 41, wherein after actuation, the bending actuator is bendable along a second direction away from the limb.

49. The limb stabilization device of claim 41, wherein the bending actuator comprises a memory foam liner.

50. The limb stabilization device of claim 41, wherein the bending actuator has a high coefficient of friction with skin, is breathable, comprises one or more blood-clotting materials, and/or is fluid-absorbent.

51. The limb stabilization device of claim 41, wherein the limb comprises a joint.

52. The limb stabilization device of claim 51, wherein upon actuation, the bending actuator generate forces to move the joint in one or multiple directions.

53. The limb stabilization device of claim 52, further comprising an inertial measurement unit for recording the angle and motion of the joint and/or a computer medium for storing the angle and motion of the joint in a digital database.

54. A limb stabilization device comprising:
a conformal material layer configured to wrap around a limb and apply a pressure to the limb at or below a threshold pressure, wherein the conformal material layer comprises a pressurizable bladder and/or a compressed memory foam to conform the conformal material layer to the limb; and
optionally at least one beam connected to the conformal material layer to support the limb.

55. The limb stabilization device of claim 54, further comprising a pressure modulator for regulating the pressure of the bladder to be at or below the threshold pressure.

56. The limb stabilization device of claim 55, wherein the pressure modulator is a check valve.

57. The limb stabilization device of claim 54, wherein the threshold pressure is equal to or below 1 psi.

58. The limb stabilization device of claim 54, wherein the beam is connected to the conformal material layer via hook and loop or the beam is connected to the conformal material layer via one or more optionally detachable mount.

59. A method of stabilizing an injured limb, comprising:
providing a limb stabilizing device of claim 1, 31, 41, or 54,
supporting the limb using the limb stabilizing device; and
pressurizing the bladder and/or releasing the compressed memory foam to conform the collar to the limb.

60. The method of claim 59, further comprising stabilizing and/or healing the limb.
